(19) United States
(12) Patent Application Publication
(10) Pub. No.: US 2022/0313656 A1
(43) Pub. Date: Oct. 6, 2022

(54) NEW CLASS OF ANTIBIOTICS HAVING LOW MIC-VALUES TOWARDS DIFFERENT STRAINS OF BACTERIA

(71) Applicant: Københavns Universitet, København K (DK)

(72) Inventors: Jørn B. Christensen, Virum (DK); Rikke Heidemann Olsen, Hvidovre (DK); Søren Wedel Svenningsen, København K (DK)

(21) Appl. No.: 17/594,575

(22) PCT Filed: Apr. 22, 2020
(86) PCT No.: PCT/EP2020/061171

§ 371 Date: Oct. 22, 2021

(30) Foreign Application Priority Data
Apr. 24, 2019 (EP) 19170886.6
Apr. 24, 2019 (EP) 19170888.2

(51) Int. Cl. A61K 31/382; A61K 31/5415; C07D 279/28; C07D 335/20; A61P 31/04

(52) U.S. Cl. CPC A61K 31/382; A61K 31/5415; C07D 279/28; C07D 335/20; A61P 31/04

(57) ABSTRACT

The present invention relates to a composition comprising a compound of formula (I) wherein X is selected from the group consisting of S, Se, P, PO, SO, NR¹, CR¹, CR¹R¹ or C₀₋₂-alkyl; Z is selected from the group consisting of hydrogen, a halogen, SR⁴, OR⁴, COR⁴ where R⁴ is a C₁₋₁₂-alkyl; each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy; d is selected from 0, 1, 2, and 3; each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S-C3-8-cycloalkyl, aryl, heteroaryl, aryloxy, heteroaryloxy, arylamino, heteroarylamino, arylalkyl, heteroarylalkyl, arylalkyloxy and heteroarylalkyloxy; e is selected from 0, 1, 2, 3, and 4; R¹ is selected from the group consisting of C₁₋₆-alkyl, C₃₋₈-cycloalkyl, aryl, heteroaryl, arylalkyl, heteroarylalkyl; R⁵ is N—(CHW)—N(Y¹)(Y²)(Y³) or C═CH—(CHW)—N(Y¹)(Y²)(Y³); each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where; Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl; Y² is selected from the group consisting of C₁₋₁₂-alkyl; Y³ is selected from the group consisting of linear or branched C₂₋₂₅-alkyl, linear or branched C₂₋₂₅ alkenyl or linear or branched C₂₋₂₅ alkynyl; where A is selected from any pharmaceutical relevant/acceptable anion/counterion; wherein if X is S and Z is a halogen then Y³ cannot be a C₂-alkyl or a branched C₃-alkyl; wherein if X is S and Z is hydrogen then Y³ cannot be C₂-alkyl or linear or branched C₅-alkyl. The invention also relates to anti-microbial composition for use as a medicament and for use in treating a microbial infection in a human subject.

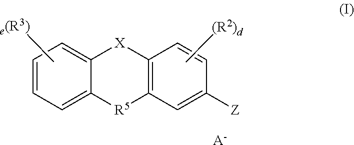

### TECHNICAL FIELD OF THE INVENTION

The present invention relates to a group of compounds having antimicrobial activity. The antibiotic compounds show low minimum inhibitory concentration (MIC) values toward a variety of bacterial strains.

### BACKGROUND OF THE INVENTION

The world is constantly seeking new antimicrobial compounds to fight the numerous infectious diseases, especially given the escalating problems associated with poorly treatable infections caused by an increasing variety of resistant infectious agents, such as antimicrobial-resistant bacteria. Antimicrobial resistance is becoming a big factor in almost all hospitals with high costs involved. WHO has recently outlined 12 bacterial species of which there is a urgent need for new antimicrobials for future treatment options.

The discovery of antibiotics has dramatically improved the public health and saved millions of lives since penicillin was first made publicly available now over 60 years ago (Aminov, R. I., “A brief history of the antibiotic era: lessons learned and challenges for the future,” Front Microbiol. 1, 134 (2010)). However, imprudent use of antibiotics have render many bacteria resistant to antibiotics, and today we are faced with the realization that the “golden year of antibiotics” may soon be replaced by the post-antibiotic era (Bryan, C. S., “Toward a post-antibiotic era?,” J. S C Med. Assoc. 91, 35 (1995) and Fowler, T., Walker, D., & Davies, S. C., “The risk/benefit of predicting a post-antibiotic era: is the alarm working?,” Ann. N. Y. Acad. Sci. 1323, 1 (2014)). New resistance mechanisms are emerging and spreading globally, threatening our ability to treat common infectious diseases, resulting in prolonged illness, disability, and death (Jones, C. A., Davis, J. S., & Looke, D. F., “Death from an untreatable infection may signal the start of the post-antibiotic era,” Med. J. Aust. 206, 292 (2017)). The human and economic costs associated with antibiotic resistance are already enormous. An increasing number of people are getting infected by resistant bacteria, which results in increased hospitalization and increased death rates (So, A. D., Gupta, N., & Cars, O., “Tackling antibiotic resistance,” BMJ 340, c2071 (2010). In Europe and the US alone antimicrobial resistant infections currently are estimated to account for approximately 50,000 deaths each year. In other parts of the world, such as the developing countries, the exact numbers remain unknown, but are most likely even higher. By 2050, resistant infections have been estimated to kill an additional 10 million people globally every year, if the current increasing antibiotic resistance trend is not counteracted (O'Neill, Report No. TRoARCbJ, 2014). Consequently, action is needed to conserve the existing antibiotics, provide new antibiotics and treatment options (So, A. D., Gupta, N., & Cars, O., “Tackling antibiotic resistance,” BMJ 340, c2071 (2010). Since the 1960s only two antibiotics belonging to new antibiotic classes have been approved (linezolid and daptomycin) (Donadio, S., et al., “Antibiotic discovery in the twenty-first century: current trends and future perspectives,” J. Antibiot. (Tokyo) 63, 423 (2010) and Roemer, T. & Boone, C., “Systems-level antimicrobial drug and drug synergy discovery,” Nat. Chem. Biol. 9, 222 (2013)). The major difficulties in bringing novel antibiotics into the market have increased the focus of so-call helper-drugs. Helper-drugs can be described as drugs that enhance the activity of antibiotics. Non-antibiotics constitute a subgroup of helper-drugs. By definition, non-antibiotics refer to drugs for which the primary approved indication(s) are non-infectious diseases (Martins, M., et al., “Potential role of non-antibiotics (helper compounds) in the treatment of multidrug-resistant Gram-negative infections: mechanisms for their direct and indirect activities,” Int. J. Antimicrob. Agents 31, 198 (2008)), but also possess bacteria modifying effects; in particularly, modification of bacterial antibiotic resistance mechanisms. Beside their antibiotic helper-drug properties, several non-antibiotic compounds have antibiotic activities on their own (Vandevelde, N. M., Tulkens, P. M., & Van, B. F., “Modulating antibiotic activity towards respiratory bacterial pathogens by co-medications: a multi-target approach,” Drug Discov. Today 21, 1114 (2016) and Kaatz, G. W., et al., “Phenylpiperidine selective serotonin reuptake inhibitors interfere with multidrug efflux pump activity in Staphylococcus aureus,” Int. J. Antimicrob. Agents 22, 254 (2003) and Hendricks, O., et al., “In vitro activity of phenothiazine derivatives in Enterococcus faecalis and Enterococcus faecium,” Basic Clin. Pharmacol. Toxicol. 96, 33 (2005)). Thioridazine, a phenothiazine derivate, is among the most intensively studied non-antibiotic compounds for reversal of resistance (Thorsing, M., et al., “Thioridazine induces major changes in global gene expression and cell wall composition in methicillin-resistant Staphylococcus aureus USA300,” PLoS. One. 8, e64518 (2013) and Deshpande, D., et al., “Thioridazine as Chemotherapy for Mycobacterium avium Complex Diseases,” Antimicrob. Agents Chemother. 60, 4652 (2016) and Amaral, L. & Viveiros, M., “Why thioridazine in combination with antibiotics cures extensively drug-resistant Mycobacterium tuberculosis infections,” Int. J. Antimicrob. Agents 39, 376 (2012) and Poulsen, M. O., et al., “Thioridazine potentiates the effect of a beta-lactam antibiotic against Staphylococcus aureus independently of mecA expression,” Res. Microbiol. 164, 181 (2013)). Thioridazine possesses inherent antibiotic activity against a broad variety of Gram-positive bacteria and in particular against Mycobacteria tuberculosis (the causative agent of tuberculosis) (van, I. J., et al., “In vitro activity of thioridazine against mycobacteria,” Int. J. Antimicrob. Agents 34, 190 (2009) and Klitgaard, J. K., et al., “Reversal of methicillin resistance in Staphylococcus aureus by thioridazine,” J. Antimicrob. Chemother. 62, 1215 (2008)). Unfortunately, the concentration of thioridazine needed for antibiotic activity is between 16-64 μl/mL, depending of bacterial species and strain. This concentration is considerably higher than what can be achieved in serum of humans or animals (Vandevelde, N. M., Tulkens, P. M., & Van, B. F., “Modulating antibiotic activity towards respiratory bacterial pathogens by co-medications: a multi-target approach,” Drug Discov. Today 21, 1114 (2016)). However, the concentration needed to modulate the antimicrobial resistance of e.g. Methicillin resistant Staphylococcus aureus (MRSA) may be as low as 4 μl/mL in vitro assays, which may be sufficient to reverse resistance of oxacillin (an important clinical antibiotic) of MRSA (Klitgaard, J. K., et al., “Reversal of methicillin resistance in Staphylococcus aureus by thioridazine,” J. Antimicrob. Chemother. 62, 1215 (2008). Depressingly, animal studies on the synergetic effect of thioridazine and dichloxacillin (an antibiotic related to oxacillin) have shown lack of synergy and severe extrapyramidal adverse effects and behavioral changes in the pigs treated with a thioridazine dose of 300 mg×2 daily. The pigs showed signs varying from dizziness and drowsiness to severe agitation with repetitive compulsive self-destructive behavior (e.g. licking or scratching the same spot of the pen walls for several hours resulting in abrasions to the head or snout) (Stenger, M., et al., “Systemic thioridazine in combination with dicloxacillin against early aortic graft infections caused by Staphylococcus aureus in a porcine model: In vivo results do not reproduce the in vitro synergistic activity,” 12, e0173362 (2017)).

Although thioridazine have shown some promising results in vitro, the main purpose has been to show that thoridazine have potential as a “helper-compound” that revers resistance. Unfortunately, as stated above, recent in vivo studies have not been able to reproduce the promising in vitro results. The discrepancy between in vitro and in vivo results is due to the lack of tolerance of thioridazine in the needed concentrations.

So, given the escalating problems associated with poorly treatable infections caused by an increasing variety of resistant infectious agents, such as antibiotic-resistant bacteria there is a great need for improved anti-microbial treatments.

Accordingly, the main object of the invention is to provide a new class of antibiotics compounds having low MIC-values towards different strains of bacteria including some resistant strains of bacteria.

### SUMMARY OF THE INVENTION

The above observations and problems with thioridazine let the inventors to speculate that a drug or class of drugs that are less permeable to the blood-brain barrier and yet sustain its antibiotic helper-drug activity could be a potential antibacterial agent. Hence, the inventors found that a specific group of compounds showed promising results.

Hence, the present disclosure relates in a first aspect to a composition comprising a compound of formula I

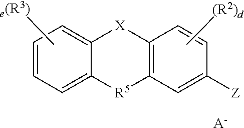

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl, linear or branched C₂₋₂₅ alkenyl or linear or branched C₂₋₂₅ alkynyl;

where A is selected from any pharmaceutical relevant/acceptable anion/counterion; wherein if X is S and Z is a halogen then Y³ cannot be a C₂-alkyl or a branched C₃-alkyl;

wherein if X is S and Z is hydrogen then Y³ cannot be C₂-alkyl or linear or branched C₅-alkyl.

It is surprising that the inventors have found out that this specific group of compounds all have antimicrobial activity towards different strains of bacteria including resistant strains. This group has shown to have low MIC-values.

The inventors managed to synthesize several modifications of thioridazine, and in addition, the same modifications were done to other phenothiazine-derivatives and to chlorprothixene.

The results shows that by modulating the chemical structure of thioridazine and of the other phenothiazine-derivatives and of chlorprothixene, the compounds were less permeable to the blood-brain barrier. Further the compounds showed between 2-128 fold higher antibiotic activities (defined by the lowest concentration of the compound that allow inhibition of bacterial growth, also known as the minimal inhibitory concentration (MIC) value) than the original hydrochloride-state of the same compound.

The present disclosure also relates in a second aspect to an anti-microbial composition comprising a compound of formula I

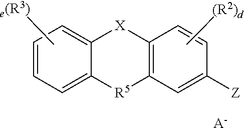

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl, linear or branched C₂₋₂₅ alkenyl or linear or branched C₂₋₂₅ alkynyl;

where A is selected from any pharmaceutical relevant/acceptable anion/counterion; for use as a medicament.

In a third aspect, the disclosure also relates to an anti-microbial composition comprising a compound of formula I

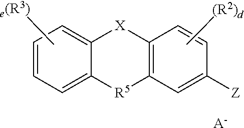

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl, linear or branched C₂₋₂₅ alkenyl or linear or branched C₂₋₂₅ alkynyl;

where A is selected from any pharmaceutical relevant/acceptable anion/counterion; for use in treating a microbial infection in a human subject.

### Definitions

In the context of the present invention, the following terms are meant to comprise the following, unless defined elsewhere in the description.

The terms “about”, “around”, or “approximately” are meant to indicate e.g. the measuring uncertainty commonly experienced in the art, which can be in the order of magnitude of e.g. +/−1, 2, 5, 10%, etc.

The term “comprising” or “to comprise” is to be interpreted as specifying the presence of the stated parts, steps, features, or components, but does not exclude the presence of one or more additional parts, steps, features, or components. E.g., a composition comprising a chemical compound may thus comprise additional chemical compounds, etc.

MIC-value defines the antibiotic activities defined by the lowest concentration of the compound that allow inhibition of bacterial growth, also known as the minimal inhibitory concentration (MIC) value.

MBC-value defines the antibiotic activities by the lowest concentration of the compound needed to kill at least 99.9% of the bacteria, also known as minimum bactericidal concentration (MBC) value.

As used herein, the term “antimicrobial composition or agent” is intended to cover drugs, chemicals, or other substances that either kill or slow the growth of microbes. Among the antimicrobial agents in use today are antibacterial drugs, antiviral agents, antifungal agents, and antiparisitic drugs. Common examples of such agents include, for example, beta-lactams (penicillins and cephalosporins), semisynthetic penicillin derivatives, clavulanic acid analogues, monobactams, carboxypenems, aminoglycosides, glycopeptides, lincomycins, macrolide antibiotics, polypeptides, polyenes, rifamycins, tetracyclines, semisynthetic tetracyclines, and chloramphenicol derivatives.

The terms “antimicrobial resistance” and “resistance” are used interchangeably to describe a situation where a pathogenic microbe has undergone some sort of change that reduces or eliminates the effectiveness of drugs, chemicals, or other agents to cure or prevent infections.

The terms “microbes” is used in its common meaning, i.e. to cover pathogenic organisms so small that a microscope is required to see them. Microbes are also called microorganisms, and include bacteria, viruses, fungi, and parasites, out of which the former two, especially bacteria are the most relevant for the purposes of the present invention.

The term “C₁₋₆-alkyl” should be understood to designate linear or branched alkyl groups comprising from 1 to 6 carbon atoms. Representative examples include methyl, ethyl, propyl, isopropyl, butyl, sec-butyl, tert-butyl, pentyl isopentyl, hexyl, methylpentyl, and neopentyl.

The term “C₁₋₁₂-alkyl” should be understood to designate linear or branched alkyl groups comprising from 1 to 12 carbon atoms. The term “C₂₋₂₅-alkyl” should be understood to designate linear or branched alkyl groups comprising from 2 to 25 carbon atoms.

The term “C₃₋₈-cycloalkyl” designates cyclic alkyl groups comprising from 3 to 8 carbon atoms, such as cyclopropyl, cyclobutyl, cyclopentyl, cyclohexyl, cyclopropylmethyl, cyclopentylethyl, methylcyclopropyl, dimethylcyclobutyl, ethylcyclopropylethyl, and the like.

The term “aryl” is intended to designate optionally substituted carbacyclic aromatic moieties, which may be isolated or fused, such as phenyl and anthryl.

The term “heteroaryl” should be understood to cover optionally substituted aromatic moieties comprising one or more heteroatoms independently selected from N, O, and S. Heteroaryl groups may further be fused to one or more heteroaryl or aryl rings so as to include bicyclic and polycyclic ring systems. The heteroaryl groups may be connected either via a heteroatom, or via a carbon atom. Preferred heteroaryl groups are those comprising the aromatic sextet, i. e. 6 pi-electrons in the ring system, and those bicyclic systems which have 10 pi-electrons. Typical examples include furyl, thienyl, pyrrolyl, indolyl, pyridyl, benzofuryl, benzothienyl, pyrazolyl, diazolyl, oxazolyl, thiazolyl, oxadiazolyl, thiadiazolyl, triazolyl, tetrazolyl, imidazolyl, benzoimidazolyl, benzoxazolyl, indazolyl, and the like.

“Arylalkyl” designates an aryl group connected through a C₁-C₆ alkylene tether such as methylene, ethylene, propylene, tetramethylene, pentamethylene, or hexamethylene.

“Heteroarylalkyl” similarly designates a heteroaryl group connected through a C₁-C₆ alkylene tether such as methylene, ethylene, propylene, tetramethylene, pentamethylene, or hexamethylene.

The terms “arylalkyloxy” and “heteroarylalkyloxy” are intended to cover arylalkyl and heteroarylalkyl groups, respectively, which are appended, as substituents, through an oxygen atom.

The terms “aryloxy”, “heteroaryloxy”, “arylamino”, and “heteroarylamino” are used in their usual meaning, i. e. aromatic or heteroaromatic groups connected, as substituents, through an oxygen atom or amino (NH) group, respectively.

“Halogen” includes fluorine, chlorine, bromine and iodine atoms.

In the context of the present invention, the term “optionally substituted” is used to incorporate the optional presence of one or more substituents which may be selected from hydroxy, C₁₋₆-alkoxy, C₂₋₆-alkenyloxy, oxo, carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, and halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidine.

Preferably, such optional substituents are selected from hydroxy, C₁₋₆-alkyl, C₁₋₆-alkoxy, carboxy, C₁₋₆-alkylcarbonyl, formyl, amino, mono- and di(C₁₋₆-alkyl)amino, carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphony-oxy, sulphanyl, amino, amino-sulfonyl, mono- and di(C₁₋₆-alkyl)amino-sulfonyl or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidine. Especially preferred examples are C₁₋₆-alkyl, C₁₋₆-alkoxy, amino, mono- and di(C₁₋₆-alkyl)amino, sulphanyl, carboxy or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-calkylcarbonylamino, halogen, C₁₋₆-alkylthio, C₁₋₆-alkyl-sulphonyl-amino, or guanidine.

### BRIEF DESCRIPTION OF THE DRAWINGS

The invention is further illustrated by the drawings, wherein:

FIG. 1 to 10: Illustrates the results as also presented in table 8 and 9.

FIG. 11: Illustrates the flux (transport) curves for the four compounds across IPEC-J2 MDR1 cell monolayers. Compound 1: 10-(2-(1-methylpiperidin-2-yl)ethyl)-2-(methylthio)-10H-phenothiazine hydrochloride (S23); compound 2: 1-ethyl-1-methyl-2-(2-(2-(methylthio)-10H-phenothiazin-10-yl)ethyl)piperidin-1-ium bromide (S25); compound 3: 1-isopentyl-1-methyl-2-(2-(2-(methylthio)-10H-phenothiazin-10-yl)ethyl)piperidin-1-ium bromide (S27); compound 4: 1-methyl-2-(2-(2-(methylthio)-10H-phenothiazin-10-yl)ethyl)-1-pentylpiperidin-1-ium bromide (S28). Values on the y-axis represents amounts of compound (nmol cm⁻²) appearing in the basolateral chamber at the time points indicated on the x-axis. Data expressed as means±SD. The figure below show the same data as the figure on the top, but with a bracketed y-axis to visualize detailed flux curves for compound 2, 3 and 4.

FIG. 12: Illustrates the calculated permeability's (Papp) for the four compounds across IPEC-J2 MDR1 cell monolayers. Compound 1: 10-(2-(1-methylpiperidin-2-yl)ethyl)-2-(methylthio)-10H-phenothiazine hydrochloride (S23); compound 2: 1-ethyl-1-methyl-2-(2-(2-(methylthio)-10H-phenothiazin-10-yl)ethyl)piperidin-1-ium bromide (S25); compound 3: 1-isopentyl-1-methyl-2-(2-(2-(methylthio)-10H-phenothiazin-10-yl)ethyl)piperidin-1-ium bromide (S27); compound 4: 1-methyl-2-(2-(2-(methylthio)-10H-phenothiazin-10-yl)ethyl)-1-pentylpiperidin-1-ium bromide (S28). Values on the y-axis represents calculated permeability (cm s⁻¹) for the four compounds. Data expressed as means±SD. The figure on the right show the same data as the figure to the left, but with a bracketed y-axis to enhance details in the bars for compound 2, 3 and 4.

FIG. 13: Shows the calculated apparent permeability (Papp) for ¹⁴C-mannitol transport across IPEC-J2 MDR1 cells monolayers exposed to either supplemented HBSS (control) or 10 μM solutions of the four compounds. Compound 1: 10-(2-(1-methylpiperidin-2-yl)ethyl)-2-(methylthio)-10H-phenothiazine hydrochloride (S23); compound 2: 1-ethyl-1-methyl-2-(2-(2-(methylthio)-1-OH-phenothiazin-10-yl)ethyl)piperidin-1-ium bromide (S25); compound 3: 1-isopentyl-1-methyl-2-(2-(2-(methylthio)-10H-phenothiazin-10-yl)ethyl)piperidin-1-ium bromide (S27); compound 4: 1-methyl-2-(2-(2-(methylthio)-10H-phenothiazin-10-yl)ethyl)-1-pentylpiperidin-1-ium bromide (S28). Values on the y-axis represents calculated permeability (cm s⁻¹) for the four compounds. Data expressed as means±SD.

FIG. 14: Shows the results of the LDH assay on samples from the different donor solutions after incubation on IPEC-J2 MDR1 cells monolayers for 120 minutes with absorbance at 492 nm as a measure for LDH release of the four compounds. Compound 1: 10-(2-(1-methylpiperidin-2-yl)ethyl)-2-(methylthio)-10H-phenothiazine hydrochloride (S23); compound 2: 1-ethyl-1-methyl-2-(2-(2-(methylthio)-10H-phenothiazin-10-yl)ethyl)piperidin-1-ium bromide (S25); compound 3: 1-isopentyl-1-methyl-2-(2-(2-(methylthio)-10H-phenothiazin-10-yl)ethyl)piperidin-1-ium bromide (S27); compound 4: 1-methyl-2-(2-(2-(methylthio)-10H-phenothiazin-10-yl)ethyl)-1-pentylpiperidin-1-ium bromide (S28). Supplemented HBSS was used as a negative control and cell lysate from IPEC-J2 MDR1 cells monolayers treated with ultrasound was used as a positive control. Values on the y-axis represents absorbance values at 492 nm as a measure of LDH release expressed as percentage relative to the absorbance obtained from donor solutions from cell monolayers exposed to the supplemented HBSS. Data expressed as means±SD.

FIG. 15: Shows the results of the absorbance measurements in supernatants of bovine whole blood incubated with different drug solutions. The four compounds are: compound TH: 10-(2-(1-methylpiperidin-2-yl)ethyl)-2-(methylthio)-10H-phenothiazine hydrochloride (S23); compound TE: 1-ethyl-1-methyl-2-(2-(2-(methylthio)-1-OH-phenothiazin-10-yl)ethyl)piperidin-1-ium bromide (S25); compound TI: 1-isopentyl-1-methyl-2-(2-(2-(methylthio)-10H-phenothiazin-10-yl)ethyl)piperidin-1-ium bromide (S27); compound TP: 1-methyl-2-(2-(2-(methylthio)-10H-phenothiazin-10-yl)ethyl)-1-pentylpiperidin-1-ium bromide (S28). The top figure show a plot of the measured absorbance at a wavelength of 540 nm. The bottom figure show the absorbance readings relative to the absorbance data measured in supernatants from red blood cells exposed to a 0.1% TritonX solution. The extend of response is proportional to degree of cell lysis. Absorbance measured in supernatants exposed to TritonX and MilliQ water is assumed to be complete (100% cell lysis), while a negligible degree of cell lysis is assumed from exposure to Phosphate buffered saline and isotonic saline.

FIG. 16: Shows the results of topical treatment twice daily for 3 days in the murine superficial MRSA skin infection model. Each data point is bacterial load in a superficial skin infection (measured as colony forming units (CFU)/ml). SOT=Start of treatment which is used to determine the bacterial load (CFU)/ml) before treatment was initiated. A 2%.: Bacterial load at day 4 (D4) for each mouse in group A treated with 2% S43-added hydrogel. A 1%: Bacterial load at day 4 (D4) for each mouse in group A treated with 1% S43-added hydrogel. Fucidin: Bacterial load at day 4 (D4) for each mouse in group A treated with 2% fusidic acid (standard treatment of Staphylococcus aureus infected wounds). Vehicle: Bacterial load at day 4 (D4) for each mouse in standard (non-antibiotic added) hydrogel.

### DETAILED DESCRIPTION OF THE INVENTION

The inventors have conducted intensive studies on a large range of compounds in an attempt to find new antibacterial drugs and has found unprecedented findings that compounds of the below given formula shows promising results in regards to having properties relevant for antimicrobial activity.

A first aspect of the invention defines a composition comprising a compound of formula I

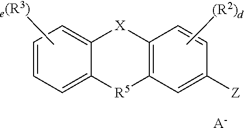

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the Wand the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl, linear or branched C₂₋₂₅ alkenyl or linear or branched C₂₋₂₅ alkynyl;

where A is selected from any pharmaceutical relevant/acceptable anion/counterion; wherein if X is S and Z is a halogen then Y³ cannot be a C₂-alkyl or a branched C₃-alkyl;

wherein if X is S and Z is hydrogen then Y³ cannot be C₂-alkyl or linear or branched C₅-alkyl.

In one embodiment, X is selected from the group consisting of S, Se, P, PO, SO, NR¹, CR¹, CR¹R² or C₀₋₂-alkyl.

It is surprising that the inventors have found out that this specific group of compounds all have antimicrobial activity towards different strain of bacteria including resistant strains.

The inventors managed to synthesize several modifications of thioridazine, and in addition, the same modifications were done to other phenothiazine-derivatives and to chlorprothixene.

The results shows that by modulating the chemical structure of thioridazine and of the other phenothiazine-derivatives and of chlorprothixene, the compounds were less permeable to the blood-brain barrier. Further the compounds showed between 2-128 fold higher antibiotic activities (defined by the lowest concentration of the compound that allow inhibition of bacterial growth, also known as the minimal inhibitory concentration (MIC) value) than the original hydrochloride-state of the same compound.

The compounds are quaternized on the side-chain nitrogen with different alkyl groups. The length of the chain has surprisingly shown to be important.

Where nothing else is mentioned or written in the definitions of the formulas herein, it is implicit that there is a hydrogen atom attached. For example; if d is selected from 0, 1 or 2, so that a R² group is positioned at 0, 1 or 2 places—there will be a hydrogen atom attached to the remaining places. Also in the formula definition for R⁵: N—(CHW)—N(Y¹)(Y²)(Y³) or C═CH—(CHW)—N(Y¹)(Y²)(Y³), it is implicit that there is a hydrogen atom attached to “fill up”, so that there won't be any radicals.

In one embodiment the compound of formula I is a phenothiazine derivative. The phenothiazine derivative can be selected from the group consisting of chlorpromazine derivatives, promethazine derivatives and thioridazine derivatives, and salts thereof.

In one embodiment the compound of formula I is a chlorprothixene derivative.

In one or more embodiment, there is at least two carbon atoms between the two nitrogen atoms. In the case where the W is a C₁-alkyl group attached to both the CH group and the N(Y¹)(Y²)(Y³) group —R⁵ is then N—(CH₂CH₂)—N(Y¹)(Y²)(Y³).

In one or more embodiment, there is at least three carbon atoms between the two nitrogen atoms. In the case where the W is a C₂-alkyl group attached to both the CH group and the N(Y¹)(Y²)(Y³) group —R⁵ is then N—(CH₂CH₂CH₂)—N(Y¹)(Y²)(Y³).

In one or more embodiment, there is at least two carbon atoms between the two nitrogen atoms. In the case where the W is a branched C₂-alkyl group attached to both the CH group and the N(Y¹)(Y²)(Y³) group —R⁵ is then N—(CH₂CH(CH₃))—N(Y¹)(Y²)(Y³).

In one or more embodiment, there is at least three carbon atoms between the carbon atom in the ring structure and the nitrogen atom. In the case where the W is a C₁-alkyl group attached to both the CH group and the N(Y¹)(Y²)(Y³) group —R⁵ is then C═CH—(CH₂CH₂)—N(Y¹)(Y²)(Y³).

In one embodiment, Y³ is a linear or branched C₂₋₂₅-alkyl. In one embodiment Y³ is a linear or branched C₂₋₂₅ alkenyl. In one embodiment, Y³ is a linear or branched C₂₋₂₅ alkynyl. In one embodiment Y³ is a linear or branched C₃₋₂₅ aliphatic group.

In one embodiment, Y³ is a linear or branched C₅₋₂₅-alkyl. In one embodiment, Y³ is a linear or branched alkyl with a side chain higher than C₅.

In one embodiment Y³ is a linear or branched C₂₋₆-alkyl. Y³ can be selected from the group consisting of ethyl, propyl, methyl-butyl, iso-propyl or pentyl.

In one embodiment Y³ is a linear or branched C₈₋₁₅-alkyl. Y³ can be selected from the group consisting of linear or branched C₈-alkyl, linear or branched C₁₀-alkyl, linear or branched C₁₂-alkyl, linear or branched C₁₄-alkyl, linear or branched C₁₅-alkyl.

In one embodiment Y¹ and Y² are individually selected from C₁₋₆-alkyl, such as from C₁₋₃-alkyl. In one embodiment Y¹ and Y² can both be C₁-alkyl.

In one embodiment Y¹ together with a carbon being part of the Wand the nitrogen atom to which it is attached forms a six-membered nitrogen-containing heterocyclyl. Y² can then in one embodiment be selected from C₁₋₃-alkyl, such as C₁-alkyl.

In one embodiment X is S. in one embodiment Z can be selected from the group consisting of hydrogen, Cl or a SR⁴ where R⁴ is a C₁-alkyl.

The pharmaceutical relevant/acceptable anion/counterion can be any suitable known relevant/acceptable anion/counterion and in one embodiment it can be selected from the group consisting of I, Br or Cl, MS or TS.

In one embodiment the compound of formula I is a chlorpromazine derivative such as N-(3-(2-chloro-10H-phenothiazin-10-yl)propyl)-N,N,3-trimethylbutan-1-aminium bromide. In one embodiment the compound is a chlorpromazine derivative such as N-(3-(2-chloro-10H-phenothiazin-10-yl)propyl)-N,N-dimethylpentan-1-aminium bromide.

In one embodiment the compound of formula I is a promethazine derivative such as N-isopropyl-N,N-dimethyl-1-(10H-phenothiazin-10-yl)propan-2-aminium bromide.

In one embodiment the compound is a chlorpromazine derivative such as N-(3-(2-chloro-10H-phenothiazin-10-yl)propyl)-N,N-dimethyldodecan-1-aminium bromide.

In one embodiment the compound is a promethazine derivative such as N-(1-(10H-phenothiazin-10-yl)propan-2-yl)-N,N-dimethyldodecan-1-aminium bromide.

In one embodiment the compound of formula I is a thioridazine derivative such as 1-ethyl-1-methyl-2-(2-(2-(methylthio)-10H-phenothiazin-10-yl)ethyl)piperidin-1-ium bromide. In one embodiment the compound is a thioridazine derivative such as 1-isopropyl-1-methyl-2-(2-(2-(methylthio)-10H-phenothiazin-10-yl)ethyl)piperidin-1-ium bromide. In one embodiment the compound is a thioridazine derivative such as 1-isopentyl-1-methyl-2-(2-(2-(methylthio)-10H-phenothiazin-10-yl)ethyl)piperidin-1-ium bromide. In one embodiment the compound is a thioridazine derivative such as 1-methyl-2-(2-(2-(methylthio)-10H-phenothiazin-10-yl)ethyl)-1-pentylpiperidin-1-ium bromide. In one embodiment the compound is a thioridazine derivative such as 1-dodecyl-1-methyl-2-(2-(2-(methylthio)-10H-phenothiazin-10-yl)ethyl)piperidin-1-ium.

In one embodiment the compound of formula I is a chlorprothixene derivative such as (Z)—N-(3-(2-chloro-9H-thioxanthen-9-ylidene)propyl)-N,N,3-trimethylbutan-1-aminium bromide. In one embodiment the compound is a chlorprothixene derivative such as (Z)—N-(3-(2-chloro-9H-thioxanthen-9-ylidene)propyl)-N,N-dimethylpentan-1-aminium bromide. In one embodiment the compound is a chlorprothixene derivative such as (Z)—N-(3-(2-chloro-9H-thioxanthen-9-ylidene)propyl)-N,N-dimethyldodecan-1-aminium bromide.

In one embodiment the compound is N-(3-(10H-phenothiazin-10-yl)propyl)-N,N,3,7-tetramethyloct-6-en-1-aminium 4-methylbenzenesulfonate.

In one embodiment the compound is N-(1-(10H-phenothiazin-10-yl)propan-2-yl)-N,N-dimethyltetradecan-1-aminium bromide.

In one embodiment the compound is N-(1-(10H-phenothiazin-10-yl)propan-2-yl)-N,N,3,7-tetramethyloct-6-en-1-aminium 4-methylbenzenesulfonate.

A second aspect of the invention defines an anti-microbial composition comprising a compound of formula I

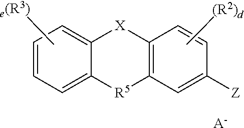

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl; linear or branched C₂₋₂₅ alkenyl or linear or branched C₂₋₂₅ alkynyl;

where A is selected from any pharmaceutical relevant/acceptable anion/counterion; for use as a medicament.

In one embodiment X is selected from the group consisting of S, Se, P, PO, SO, NR¹, CR¹, CR¹R² or C₀₋₂-alkyl.

In one embodiment of the second aspect, the compound of formula I in the anti-microbial composition is a phenothiazine derivative. The phenothiazine derivative can be selected from the group consisting of chlorpromazine derivatives, promethazine derivatives and thioridazine derivatives, and salts thereof. In one embodiment the compound of formula I is a chlorprothixene derivative.

In one or more embodiment, there is at least two carbon atoms between the two nitrogen atoms. In the case where the W is a C₁-alkyl group attached to both the CH group and the N(Y¹)(Y²)(Y³) group —R⁵ is then N—(CH₂CH₂)—N(Y¹)(Y²)(Y³).

In one or more embodiment, there is at least three carbon atoms between the two nitrogen atoms. In the case where the W is a C₂-alkyl group attached to both the CH group and the N(Y¹)(Y²)(Y³) group —R⁵ is then N—(CH₂CH₂CH₂)—N(Y¹)(Y²)(Y³).

In one or more embodiment, there is at least two carbon atoms between the two nitrogen atoms. In the case where the W is a branched C₂-alkyl group attached to both the CH group and the N(Y¹)(Y²)(Y³) group —R⁵ is then N—(CH₂CH(CH₃))—N(Y¹)(Y²)(Y³).

In one or more embodiment, there is at least three carbon atoms between the carbon atom in the ring structure and the nitrogen atom. In the case where the W is a C₁-alkyl group attached to both the CH group and the N(Y¹)(Y²)(Y³) group —R⁵ is then C═CH—(CH₂CH₂)—N(Y¹)(Y²)(Y³).

In one embodiment, Y³ is a linear or branched C₂₋₂₅-alkyl. In one embodiment Y³ is a linear or branched C₂₋₂₅ alkenyl. In one embodiment, Y³ is a linear or branched C₂₋₂₅ alkynyl. In one embodiment Y³ is a linear or branched C₃₋₂₅ aliphatic group.

In one embodiment, Y³ is a linear or branched C₅₋₂₅-alkyl. In one embodiment, Y³ is a linear or branched alkyl with a side chain higher than C₅.

In one embodiment Y³ is a linear or branched C₂₋₆-alkyl. Y³ can be selected from the group consisting of ethyl, propyl, methyl-butyl, iso-propyl or pentyl. In one embodiment Y¹ and Y² are individually selected from C₁₋₆-alkyl, such as from C₁₋₃-alkyl. In one embodiment Y¹ and Y² can both be C₁-alkyl. In one embodiment Y³ is a linear or branched C₂₋₆-alkyl. Y³ can be selected from the group consisting of ethyl, propyl, methyl-butyl, iso-propyl or pentyl. Y¹ and Y² can be individually selected from C₁₋₆-alkyl, such as from C₁₋₃-alkyl. Y¹ and Y² can both be C₁-alkyl in one embodiment.

In one embodiment Y³ is a linear or branched C₈₋₁₅-alkyl. Y³ can be selected from the group consisting of linear or branched C₈-alkyl, linear or branched C₁₀-alkyl, linear or branched C₁₂-alkyl, linear or branched C₁₄-alkyl, linear or branched C₁₅-alkyl. In one embodiment Y¹ and Y² are individually selected from C₁₋₆-alkyl, such as from C₁₋₃-alkyl. In one embodiment Y¹ and Y² can both be C₁-alkyl. In one embodiment Y³ is a linear or branched C₈₋₁₂-alkyl. Y³ can be selected from the group consisting of linear or branched C₈-alkyl, linear or branched C-alkyl, linear or branched C₁₀-alkyl, linear or branched C₁₁-alkyl or linear or branched C₁₂-alkyl. Y¹ and Y² can be individually selected from C₁₋₆-alkyl, such as from C₁₋₃-alkyl. Y¹ and Y² can both be C₁-alkyl in one embodiment.

In one embodiment the anti-microbial composition comprise that Y¹ together with a carbon being part of the Wand the nitrogen atom to which it is attached forms a six-membered nitrogen-containing heterocyclyl and Y² can be selected from C₁₋₃-alkyl, such as C₁-alkyl.

In one embodiment of the anti-microbial composition X is S. In one embodiment Z is selected from the group consisting of hydrogen, C₁ or a SR⁴ where R⁴ is a C₁-alkyl.

The anti-microbial composition for use as a medicament according to the second aspect can have antibacterial properties against resistant strains of bacteria (resistant towards conventional antimicrobial). These resistant strains of bacteria can be selected from the genus of Staphylococcus, Bacillus, Enterococcus, Streptococcus, Listeria, Escherichia or Salmonella. The resistant strains of bacteria can also be selected from the genus of Clostridium, Cutibacterium or Campylobacter.

Hence, the anti-microbial composition for use as a medicament according to the second aspect can be used to treat an infection, which is caused by the selected bacteria.

The anti-microbial composition for use as a medicament according to the second aspect can have antibacterial properties against Gram-positive bacteria. These Gram-positive bacteria can be selected from the genus of Staphylococcus, Bacillus, Enterococcus, Streptococcus, Listeria or Cutibacterium, such as selected from Staphylococcus aureus, Bacillus cereus, Enterococcus faecium, Enterococcus faecalis, Staphylococcus pseudintermedius, Staphylococcus epidemidis, Streptococcus equi, Cutibacterium acnes, Clostridium difficile, Listeria monocytogenes strains.

The anti-microbial composition for use as a medicament according to the second aspect can have antibacterial properties against certain Gram-negative bacteria. These Gram-negative bacteria can be selected from the genus of Campylobacter, Escherichia or Salmonella such as selected from Campylobacter jejuni, Escherichia coli or Salmonella Enteritidis strains.

The anti-microbial composition for use as a medicament according to the second aspect can have antibacterial properties against Staphylococcus epidermidis.

The anti-microbial composition for use as a medicament according to the second aspect can have antibacterial properties against Campylobacter jejuni.

The anti-microbial composition for use as a medicament according to the second aspect can have antibacterial properties against Cutibacterium acnes.

The anti-microbial composition for use as a medicament according to the second aspect can have antibacterial properties against multi-resistant strains of bacteria. The multi-resistant strains of bacteria can be selected from Staphylococcus aureus Bacillus cereus, Staphylococcus epidermidis, Clostridium difficile, Enterococcus faecalis, or Enterococcus faecium.

The anti-microbial composition for use as a medicament according to the present disclosure can have a minimum inhibitory concentration (MIC) below 16 μg/mL, such as below 8 μg/mL, such as below 4 μg/mL or such as below 2 μg/mL.

In one embodiment of the second aspect the compound of formula I is a chlorpromazine derivative such as N-(3-(2-chloro-10H-phenothiazin-10-yl)propyl)-N,N,3-trimethylbutan-1-aminium bromide. In one embodiment of the second aspect the compound is a chlorpromazine derivative such as N-(3-(2-chloro-10H-phenothiazin-10-yl)propyl)-N,N-dimethylpentan-1-aminium bromide. In one embodiment of the second aspect the compound is a chlorpromazine derivative such as N-(3-(2-chloro-10H-phenothiazin-10-yl)propyl)-N,N-dimethyldodecan-1-aminium bromide.

In one embodiment of the second aspect the compound of formula I is a promethazine derivative such as N-isopropyl-N,N-dimethyl-1-(10H-phenothiazin-10-yl)propan-2-aminium bromide. In one embodiment of the second aspect the compound is a promethazine derivative such as N-(1-(10H-phenothiazin-10-yl)propan-2-yl)-N,N-dimethyldodecan-1-aminium bromide.

In one embodiment of the second aspect the compound of formula I is a thioridazine derivative such as 1-ethyl-1-methyl-2-(2-(2-(methylthio)-1-OH-phenothiazin-10-yl)ethyl)piperidin-1-ium bromide. In one embodiment of the second aspect the compound is a thioridazine derivative such as 1-isopropyl-1-methyl-2-(2-(2-(methylthio)-10H-phenothiazin-10-yl)ethyl)piperidin-1-ium bromide. In one embodiment of the second aspect the compound is a thioridazine derivative such as 1-isopentyl-1-methyl-2-(2-(2-(methylthio)-10H-phenothiazin-10-yl)ethyl)piperidin-1-ium bromide. In one embodiment of the second aspect the compound is a thioridazine derivative such as 1-methyl-2-(2-(2-(methylthio)-10H-phenothiazin-10-yl)ethyl)-1-pentylpiperidin-1-ium bromide. In one embodiment of the second aspect the compound is a thioridazine derivative such as 1-dodecyl-1-methyl-2-(2-(2-(methylthio)-10H-phenothiazin-10-yl)ethyl)piperidin-1-ium bromide.

In one embodiment of the second aspect the compound of formula I is a chlorprothixene derivative such as (Z)—N-(3-(2-chloro-9H-thioxanthen-9-ylidene)propyl)-N,N,3-trimethylbutan-1-aminium bromide. In one embodiment of the second aspect the compound is a chlorprothixene derivative such as (Z)—N-(3-(2-chloro-9H-thioxanthen-9-ylidene)propyl)-N,N-dimethylpentan-1-aminium bromide. In one embodiment of the second aspect the compound is a chlorprothixene derivative such as (Z)—N-(3-(2-chloro-9H-thioxanthen-9-ylidene)propyl)-N,N-dimethyldodecan-1-aminium bromide.

In one embodiment the compound is N-(3-(10H-phenothiazin-10-yl)propyl)-N,N,3,7-tetramethyloct-6-en-1-aminium 4-methylbenzenesulfonate.

In one embodiment the compound is N-(1-(10H-phenothiazin-10-yl)propan-2-yl)-N,N-dimethyltetradecan-1-aminium bromide.

In one embodiment the compound is N-(1-(10H-phenothiazin-10-yl)propan-2-yl)-N,N,3,7-tetramethyloct-6-en-1-aminium 4-methylbenzenesulfonate.

A third aspect of the invention defines an anti-microbial composition comprising a compound of formula I

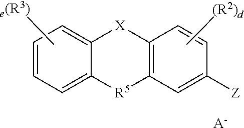

wherein

X is selected from the group consisting of S, Se, P, PO, SO, NR¹, CR¹, CR¹R¹ or C₀₋₂-alkyl;

Z is selected from the group consisting of hydrogen, a halogen, SR⁴, OR⁴, COR⁴ where R⁴ is a C₁₋₁₂-alkyl;

each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

d is selected from 0, 1, 2, and 3;

each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;

e is selected from 0, 1, 2, 3, and 4;

R¹ is selected from the group consisting of C₁₋₆-alkyl, C₃₋₈-cycloalkyl, aryl, heteroaryl, arylalkyl, heteroarylalkyl;

R⁵ is N—(CHW)—N(Y¹)(Y²)(Y³) or C═CH—(CHW)—N(Y¹)(Y²)(Y³);

each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;

Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;

Y² is selected from the group consisting of C₁₋₁₂-alkyl;

Y³ is selected from the group consisting of linear or branched C₂₋₂₅-alkyl, Hear or branched C₂₋₂₅ alkenyl or linear or branched C₂₋₂₅ alkynyl;

where A is selected from any pharmaceutical relevant/acceptable anion/counterion; for use in treating a microbial infection in a human subject.

In one embodiment X is selected from the group consisting of S, Se, P, PO, SO, NR¹, CR¹, CR¹R² or C₀₋₂-alkyl.

In one embodiment of the third aspect the compound of formula I in the anti-microbial composition is a phenothiazine derivative. The phenothiazine derivative can be selected from the group consisting of chlorpromazine derivatives, promethazine derivatives and thioridazine derivatives, and salts thereof. In one embodiment the compound of formula I is a chlorprothixene derivative.

In one or more embodiment, there is at least two carbon atoms between the two nitrogen atoms. In the case where the W is a C₁-alkyl group attached to both the CH group and the N(Y¹)(Y²)(Y³) group —R⁵ is then N—(CH₂CH₂)—N(Y¹)(Y²)(Y³).

In one or more embodiment, there is at least three carbon atoms between the two nitrogen atoms. In the case where the W is a C₂-alkyl group attached to both the CH group and the N(Y¹)(Y²)(Y³) group —R⁵ is then N—(CH₂CH₂CH₂)—N(Y¹)(Y²)(Y³).

In one or more embodiment, there is at least two carbon atoms between the two nitrogen atoms. In the case where the W is a branched C₂-alkyl group attached to both the CH group and the N(Y¹)(Y²)(Y³) group —R⁵ is then N—(CH₂CH(CH₃))—N(Y¹)(Y²)(Y³).

In one or more embodiment, there is at least three carbon atoms between the carbon atom in the ring structure and the nitrogen atom. In the case where the W is a C₁-alkyl group attached to both the CH group and the N(Y¹)(Y²)(Y³) group —R⁵ is then C═CH—(CH₂CH₂)—N(Y¹)(Y²)(Y³).

In one embodiment, Y³ is a linear or branched C₂₋₂₅-alkyl. In one embodiment Y³ is a linear or branched C₂₋₂₅ alkenyl. In one embodiment, Y³ is a linear or branched C₂₋₂₅ alkynyl. In one embodiment Y³ is a linear or branched C₃₋₂₅ aliphatic group.

In one embodiment, Y³ is a linear or branched C₅₋₂₅-alkyl. In one embodiment, Y³ is a linear or branched alkyl with a side chain higher than C₅.

In one embodiment Y³ is a linear or branched C₂₋₆-alkyl. Y³ can be selected from the group consisting of ethyl, propyl, methyl-butyl, iso-propyl or pentyl. In one embodiment Y¹ and Y² are individually selected from C₁₋₆-alkyl, such as from C₁₋₃-alkyl. In one embodiment Y¹ and Y² can both be C₁-alkyl. In one embodiment Y³ is a linear or branched C₂₋₆-alkyl. Y³ can be selected from the group consisting of ethyl, propyl, methyl-butyl, iso-propyl or pentyl. Y¹ and Y² can be individually selected from C₁₋₆-alkyl, such as from C₁₋₃-alkyl. Y¹ and Y² can both be C₁-alkyl in one embodiment.

In one embodiment Y³ is a linear or branched C₈₋₁₅-alkyl. Y³ can be selected from the group consisting of linear or branched C₈-alkyl, linear or branched C₁₀-alkyl, linear or branched C₁₂-alkyl, linear or branched C₁₄-alkyl, linear or branched C₁₅-alkyl. In one embodiment Y¹ and Y² are individually selected from C₁₋₆-alkyl, such as from C₁₋₃-alkyl. In one embodiment Y¹ and Y² can both be C₁-alkyl. In one embodiment Y³ is a linear or branched C₈₋₁₂-alkyl. Y³ can be selected from the group consisting of linear or branched C₈-alkyl, linear or branched C-alkyl, linear or branched C₁₀-alkyl, linear or branched C₁₁-alkyl or linear or branched C₁₂-alkyl. Y¹ and Y² can be individually selected from C₁₋₆-alkyl, such as from C₁₋₃-alkyl. Y¹ and Y² can both be C₁-alkyl in one embodiment.

In one embodiment the anti-microbial composition comprise that Y¹ together with a carbon being part of the W and the nitrogen atom to which it is attached forms a six-membered nitrogen-containing heterocyclyl and Y² can be selected from C₁₋₃-alkyl, such as C₁-alkyl.

In one embodiment of the anti-microbial composition X is S. In one embodiment Z is selected from the group consisting of hydrogen, C₁ or a SR⁴ where R⁴ is a C₁-alkyl.

The anti-microbial composition for use as a medicament according to the third aspect can have antibacterial properties against resistant (towards conventional antimicrobials) and non-resistant strains of bacteria. The resistant strains of bacteria can be selected from the genus of Staphylococcus, Bacillus, Enterococcus, Streptococcus, Listeria, Escherichia or Salmonella. The resistant strains of bacteria can also be selected from the genus of Clostridium, Cutibacterium or Campylobacter.

Hence, the anti-microbial composition for use as a medicament according to the third aspect can be used to treat an infection, which is caused by the selected bacteria.

The anti-microbial composition for use as a medicament according to the third aspect can have antibacterial properties against Gram-positive bacteria. These Gram-positive bacteria can be selected from the genus of Staphylococcus, Bacillus, Enterococcus, Streptococcus, Listeria or Cutibacterium, such as selected from Staphylococcus aureus, Bacillus cereus, Enterococcus faecium, Enterococcus faecalis, Staphylococcus pseudintermedius, Staphylococcus epidemidis, Cutibacterium acnes, Streptococcus equi, Clostridium difficile, Listeria monocytogenes strains.

The anti-microbial composition for use as a medicament according to the third aspect can have antibacterial properties against certain Gram-negative bacteria. These Gram-negative bacteria can be selected from the genus of Campylobacter, Escherichia or Salmonella such as selected from Campylobacter jejuni, Escherichia coli or Salmonella Enteritidis strains.

The anti-microbial composition for use as a medicament according to the third aspect can have antibacterial properties against Staphylococcus epidermidis.

The anti-microbial composition for use as a medicament according to the third aspect can have antibacterial properties against Campylobacter jejuni.

The anti-microbial composition for use as a medicament according to the third aspect can have antibacterial properties against Cutibacterium acnes.

The anti-microbial composition for use as a medicament according to the third aspect can have antibacterial properties against multi-resistant strains of bacteria. The multi-resistant strains of bacteria can be selected from Staphylococcus aureus, Bacillus cereus, Staphylococcus epidermidis, Clostridium difficile, Enterococcus faecalis, or Enterococcus faecium.

The anti-microbial composition for use as a medicament according to the present disclosure can have a minimum inhibitory concentration (MIC) below 16 μg/mL, such as below 8 μg/mL, such as below 4 μg/mL or such as below 2 μg/mL.

In one embodiment of the third aspect the compound of formula I is a chlorpromazine derivative such as N-(3-(2-chloro-10H-phenothiazin-10-yl)propyl)-N,N,3-trimethylbutan-1-aminium bromide. In one embodiment of the third aspect the compound is a chlorpromazine derivative such as N-(3-(2-chloro-10H-phenothiazin-10-yl)propyl)-N,N-dimethylpentan-1-aminium bromide. In one embodiment of the third aspect the compound is a chlorpromazine derivative such as N-(3-(2-chloro-10H-phenothiazin-10-yl)propyl)-N,N-dimethyldodecan-1-aminium bromide.

In one embodiment of the third aspect the compound of formula I is a promethazine derivative such as N-isopropyl-N,N-dimethyl-1-(10H-phenothiazin-10-yl)propan-2-aminium bromide. In one embodiment of the third aspect the compound is a promethazine derivative such as N-(1-(10H-phenothiazin-10-yl)propan-2-yl)-N,N-dimethyldodecan-1-aminium bromide.

In one embodiment of the third aspect the compound of formula I is a thioridazine derivative such as 1-ethyl-1-methyl-2-(2-(2-(methylthio)-10H-phenothiazin-10-yl)ethyl)piperidin-1-ium bromide. In one embodiment of the third aspect the compound is a thioridazine derivative such as 1-isopropyl-1-methyl-2-(2-(2-(methylthio)-10H-phenothiazin-10-yl)ethyl)piperidin-1-ium bromide. In one embodiment of the third aspect the compound is a thioridazine derivative such as 1-isopentyl-1-methyl-2-(2-(2-(methylthio)-10H-phenothiazin-10-yl)ethyl)piperidin-1-ium bromide. In one embodiment of the third aspect the compound is a thioridazine derivative such as 1-methyl-2-(2-(2-(methylthio)-10H-phenothiazin-10-yl)ethyl)-1-pentylpiperidin-1-ium bromide. In one embodiment of the third aspect the compound is a thioridazine derivative such as 1-dodecyl-1-methyl-2-(2-(2-(methylthio)-10H-phenothiazin-10-yl)ethyl)piperidin-1-ium bromide.

In one embodiment of the third aspect the compound of formula I is a chlorprothixene derivative such as (Z)—N-(3-(2-chloro-9H-thioxanthen-9-ylidene)propyl)-N,N,3-trimethylbutan-1-aminium bromide. In one embodiment of the third aspect the compound is a chlorprothixene derivative such as (Z)—N-(3-(2-chloro-9H-thioxanthen-9-ylidene)propyl)-N,N-dimethylpentan-1-aminium bromide. In one embodiment of the third aspect the compound is a chlorprothixene derivative such as (Z)—N-(3-(2-chloro-9H-thioxanthen-9-ylidene)propyl)-N,N-dimethyldodecan-1-aminium bromide.

In one embodiment the compound is N-(3-(10H-phenothiazin-10-yl)propyl)-N,N,3,7-tetramethyloct-6-en-1-aminium 4-methylbenzenesulfonate.

In one embodiment the compound is N-(1-(10H-phenothiazin-10-yl)propan-2-yl)-N,N-dimethyltetradecan-1-aminium bromide.

In one embodiment the compound is N-(1-(10H-phenothiazin-10-yl)propan-2-yl)-N,N,3,7-tetramethyloct-6-en-1-aminium 4-methylbenzenesulfonate.

In one embodiment the microbial infection can be a topical or intestinal infection. In one embodiment, the topical infection can be acne.

As example 7 shows, the compound 43 was evaluated for efficacy in an MRSA skin infection model and showed to be highly effective in decreasing the bacterial load of MRSA.

### EXAMPLES

All analytical values specified as ratio and in percent are by weight.

### Example 1

General procedure for removal of HCl salt:

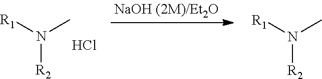

The tertiary amine hydrochloride salt of Chlorpromazine, Thioridazine, Promethazine or Promazine (2.00 g; 4.91 mmol-6.25 mmol) was added to a round bottomed flask equipped with a magnetic stirring bar. Et20 (40 ml) and NaOH (2M) (40 ml) was added and the two phase system was stirred vigorously overnight. The two phases were separated and the water phase was extracted with ether (50 ml). The combined ether phase was dried over MgSO₄, filtered and evaporated in vacuo giving the free tertiary amine.

Chlorpromazine: Yield: 1.55 g (87%)

Thioridazine: Yield: 1.71 g (94%)

Promethazine: Yield: 1.65 g (93%)

Promazine: Yield: 1.68 g (95%)

### Example 2

General synthesis procedure for the quaternization of the tertiary amine:

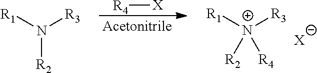

The free tertiary amine was added to a round bottomed flask equipped with a magnetic stirring bar. A solution of the alkyl halide in acetonitrile was added and refluxed in the dark overnight. The solution was evaporated in vacuo and dissolved in a min. of methanol. The methanol solution was dropped out in diethyl ether with stirring. The suspension was stirred until clear ether phase, and the ether was decanted off. Residues of ether were removed from the precipitate by high vacuum.

All the synthesized compounds are verified by either NMR and/or by HRMS (not shown).

Chlorpromazine hydrochloride (S1) (see table 1) is the starting material bought from commercial supplier.

3-(2-chloro-10H-phenothiazin-10-yl)-N,N,N-trimethylpropan-1-aminium iodide (S2):

Chlorpromazine (1.00 g; 3.14 mmol), Methyl iodide (2.0 ml; 32 mmol), Acetonitrile (18 ml), Diethyl ether (200 ml), Yield: 1.44 g (99%)

3-(2-chloro-10H-phenothiazin-10-yl)-N-ethyl-N,N-dimethylpropan-1-aminium bromide (S3):

Chlorpromazine (1.00 g; 3.14 mmol), Ethyl bromide (2.0 ml; 27 mmol), Acetonitrile (18 ml), Diethyl ether (200 ml), Yield: 1.25 g (93%)

3-(2-chloro-10H-phenothiazin-10-yl)-N-isopropyl-N,N-dimethylpropan-1-aminium bromide (S4):

Chlorpromazine (0.50 g; 1.6 mmol),2-Bromopropane (4.0 ml; 43 mmol), Acetonitrile (16 ml), Diethyl ether (200 ml), Yield: 0.69 g (100%)

N-(3-(2-chloro-10H-phenothiazin-10-yl)propyl)-N,N,3-trimethylbutan-1-aminium bromide (S5):

Chlorpromazine (0.48 g; 1.15 mmol), 1-bromo-3-methylbutane (2.0 ml; 16 mmol), Acetonitrile (18 ml), Diethyl ether (200 ml), Yield: 0.54 g (76%)

N-(3-(2-chloro-10H-phenothiazin-10-yl)propyl)-N,N-dimethylpentan-1-aminium bromide (S6):

Chlorpromazine (0.38 g; 1.19 mmol), 1-bromopentane (2.0 ml; 16 mmol), Acetonitrile (18 ml), Diethyl ether (200 ml), Yield: 0.16 g (29%)

N-(3-(2-chloro-10H-phenothiazin-10-yl)propyl)-N,N-dimethyldodecan-1-aminium bromide (S7):

Chlorpromazine (0.56 g; 1.76 mmol), 1-bromododecane (3.0 ml; 13 mmol), Acetonitrile (17 ml).

After reflux, the solution was concentrated in vacuo and purified by a silica plug (Silica: 60 Å, 15-40 μm). (Eluent: Heptane, released with DCM/MeOH (1:1)). The solution was evaporated in vacuo giving the wanted compound. Yield: 0.91 g (91%)

Promethazine hydrochloride (S12) (see table 2) is the starting material bought from commercial supplier.

N,N,N-trimethyl-1-(10H-phenothiazin-10-yl)propan-2-aminium iodide (S13):

Promethazine (1.00 g; 3.52 mmol) Methyl iodide (2.0 ml; 32 mmol), Acetonitrile (18 ml), Diethyl ether (200 ml), Yield: 1.48 g (99%)

N-ethyl-N,N-dimethyl-1-(10H-phenothiazin-10-yl)propan-2-aminium bromide (S14):

Promethazine (1.00 g; 3.52 mmol) Ethyl bromide (2.0 ml; 27 mmol), Acetonitrile (18 ml), Diethyl ether (200 ml), Yield: 1.03 g (94%)

N-isopropyl-N,N-dimethyl-1-(10H-phenothiazin-10-yl)propan-2-aminium bromide (S15):

Promethazine (0.50 g; 1.8 mmol) 2-Bromopropane (4.0 ml; 43 mmol), Acetonitrile (16 ml), Diethyl ether (200 ml), Yield: 0.10 g (14%)

N-(1-(10H-phenothiazin-10-yl)propan-2-yl)-N,N,3-trimethylbutan-1-aminium bromide (S16):

Promethazine (0.57 g; 2.00 mmol), 1-bromo-3-methylbutane (2.0 ml; 16 mmol), Acetonitrile (18 ml), Diethyl ether (200 ml), Yield: 0.16 g (68%)

N-(1-(10H-phenothiazin-10-yl)propan-2-yl)-N,N-dimethylpentan-1-aminium bromide (S17):

Promethazine (0.64 g; 2.25 mmol), 1-bromopentane (2.0 ml; 16 mmol), Acetonitrile (18 ml), Diethyl ether (200 ml), Yield: 0.91 g (93%)

N-(1-(10H-phenothiazin-10-yl)propan-2-yl)-N,N-dimethyldodecan-1-aminium bromide (S18):

Promethazine (0.78 g; 2.74 mmol), 1-bromododecane (3.0 ml; 13 mmol), Acetonitrile (17 ml). After reflux, the solution was concentrated in vacuo and purified by a silica plug (Silica: 60 Å, 15-40 μm). (Eluent: Heptane, released with DCM/MeOH (1:1)). The solution was evaporated in vacuo giving the wanted compound. Yield: 1.37 g (94%)

Thioridazine hydrochloride (S23) (see table 3) is the starting material bought from commercial supplier.

1,1-dimethyl-2-(2-(2-(methylthio)-10H-phenothiazin-10-yl)ethyl)piperidin-1-ium iodide (S24):

Thioridazine (1.00 g; 2.70 mmol) Methyl iodide (2.0 ml; 32 mmol), Acetonitrile (18 ml), Diethyl ether (200 ml), Yield: 1.37 g (99%)

1-ethyl-1-methyl-2-(2-(2-(methylthio)-10H-phenothiazin-10-yl)ethyl)piperidin-1-ium bromide (S25):

Thioridazine (1.00 g; 2.70 mmol) Ethyl bromide (2.0 ml; 27 mmol), Acetonitrile (18 ml), Diethyl ether (200 ml), Yield: 1.24 g (96%)

1-isopropyl-1-methyl-2-(2-(2-(methylthio)-10H-phenothiazin-10-yl)ethyl)piperidin-1-ium bromide (S26):

Thioridazine (0.50 g; 1.3 mmol) 2-Bromopropane (4.0 ml; 43 mmol), Acetonitrile (16 ml), Diethyl ether (200 ml), Yield: 0.09 g (13%)

1-isopentyl-1-methyl-2-(2-(2-(methylthio)-10H-phenothiazin-10-yl)ethyl)piperidin-1-ium bromide (S27):

Thioridazine (0.55 g; 1.48 mmol), 1-bromo-3-methylbutane (2.0 ml; 16 mmol), Acetonitrile (18 ml), Diethyl ether (200 ml), Yield: 0.16 g (27%)

1-methyl-2-(2-(2-(methylthio)-10H-phenothiazin-10-yl)ethyl)-1-pentylpiperidin-1-ium bromide (S28):

Thioridazine (0.62 g; 1.67 mmol), 1-bromopentane (2.0 ml; 16 mmol), Acetonitrile (18 ml), Diethyl ether (200 ml), Yield: 0.44 g (51%)

1-dodecyl-1-methyl-2-(2-(2-(methylthio)-1-OH-phenothiazin-10-yl)ethyl)piperidin-1-ium bromide (S29):

Thioridazine (0.90 g; 2.43 mmol), 1-bromododecane (3.0 ml; 13 mmol), Acetonitrile (17 ml). After reflux, the solution was concentrated in vacuo and purified by a silica plug (Silica: 60 Å, 15-40 μm). (Eluent: Heptane, released with DCM/MeOH (1:1)). The solution was evaporated in vacuo giving the wanted compound. Yield: 1.50 g (99%)

Chlorprothixene (S34) (see table 4) is the starting material bought from commercial supplier.

(Z)-3-(2-chloro-9H-thioxanthen-9-ylidene)-N,N,N-trimethylpropan-1-aminium iodide (S35):

Chlorprothixene (0.15 g; 0.47 mmol), Methyl iodide (2.0 ml; 32 mmol), Acetonitrile (18 ml), Diethyl ether (100 ml), Yield: 0.22 g (100%) (Alkyl halide: methyl iodide)

(Z)-3-(2-chloro-9H-thioxanthen-9-ylidene)-N-ethyl-N,N-dimethylpropan-1-aminium bromide (S36):

Chlorprothixene (0.15 g; 0.47 mmol), Ethyl bromide (2.0 ml; 27 mmol), Acetonitrile (18 ml), Diethyl ether (100 ml), Yield: 0.20 g (100%) (Alkyl halide: Ethyl bromide)

(Z)—N-(3-(2-chloro-9H-thioxanthen-9-ylidene)propyl)-N,N,3-trimethylbutan-1-aminium bromide (S37):

Chlorprothixene (0.15 g; 0.47 mmol), 1-bromo-3-methylbutane (2.0 ml; 16 mmol), Acetonitrile (18 ml), Diethyl ether (100 ml), Yield: 0.22 g (100%) (Alkyl halide: Pentyl bromide)

Z)—N-(3-(2-chloro-9H-thioxanthen-9-ylidene)propyl)-N,N-dimethylpentan-1-aminium bromide (S38):

Chlorprothixene (0.15 g; 0.47 mmol), 1-bromopentane (2.0 ml; 16 mmol), Acetonitrile (18 ml), Diethyl ether (100 ml), Yield: 0.22 g (100%) (Alkyl halide: 1-Bromo-3-methylbutane)

(Z)—N-(3-(2-chloro-9H-thioxanthen-9-ylidene)propyl)-N,N-dimethyldodecan-1-aminium bromide (39):

Chlorprothixene (0.05 g; 0.16 mmol), 1-bromododecane (1.0 ml; 4.2 mmol), Acetonitrile (9 ml). After reflux, the solution was concentrated in vacuo and purified by a silica plug (Silica: 60 Å, 15-40 μm). (Eluent: Heptane, released with DCM/MeOH (1:1)). The solution was evaporated in vacuo giving the wanted compound. Yield: 0.09 g (100%)

N-(3-(2-chloro-10H-phenothiazin-10-yl)propyl)-N,N-dimethylprop-2-en-1-aminium bromide (S40):

Chlorpromazine (0.20 g; 0.63 mmol), allyl bromide (2.0 ml; 23 mmol), Acetonitrile (18 ml), Diethyl ether (100 ml), Yield: 0.27 g (96%).

N-(1-(10H-phenothiazin-10-yl)propan-2-yl)-N,N-dimethylprop-2-en-1-aminium bromide (S41):

Promethazine (0.20 g; 0.70 mmol), Allyl bromide (2.0 ml; 23 mmol), Acetonitrile (18 ml), Diethyl ether (100 ml), Yield: 0.25 g (86%)

1-allyl-1-methyl-2-(2-(2-(methylthio)-10H-phenothiazin-10-yl)ethyl)piperidin-1-ium bromide (S42):

Thioridazine (0.20 g; 0.54 mmol), Allyl bromide (2.0 ml; 23 mmol), Acetonitrile (18 ml), Diethyl ether (100 ml), Yield: 0.23 g (85%).

N-(3-(10H-phenothiazin-10-yl)propyl)-N,N,3,7-tetramethyloct-6-en-1-aminium 4-methylbenzenesulfonate (S43):

Promazine (1.0 g; 3.3 mmol), 3,7-Dimethyloct-6-en-1-yl 4-methylbenzenesulfonate (1.2 g; 3.8 mmol) (synthesized by the procedure of P. H. G. Zarbin, A. Rreckziegel, E. Plass, M. Borges, W. Francke, Journal of Chemical Ecology, 26, 2737-2746 (2000)), Acetonitrile (25 ml). Heated to +40° C. for 5 days. Removed the acetonitrile in vacuo and added diethyl ether (50 ml) to the residue. Yield: 1.2 g (58%).

N-(1-(10H-phenothiazin-10-yl)propan-2-yl)-N,N-dimethyltetradecan-1-aminium bromide (S44):

Promethazine (1.1 g; 3.7 mmol); 1-Bromotetradecane (0.91 g; 3.3 mmol); Acetonitrile (25 ml). Heated to +40° C. for 7 days. Removed the acetonitrile in vacuo and added petroleum ether Bp<50° C. (50 ml) to the residue. Yield: 1.2 g (60%).

N-(1-(10H-phenothiazin-10-yl)propan-2-yl)-N,N,3,7-tetramethyloct-6-en-1-aminium 4-methylbenzenesulfonate (S45):

Promethazine (0.97 g; 3.4 mmol); 3,7-Dimethyloct-6-en-1-yl 4-methylbenzenesulfonate (1.2 g; 3.8 mmol) (synthesized by the procedure of P. H. G. Zarbin, A. Rreckziegel, E. Plass, M. Borges, W. Francke, Journal of Chemical Ecology, 26, 2737-2746 (2000)), Acetonitrile (25 ml). Heated to +40° C. for 7 days. Removed the acetonitrile in vacuo and added diethyl ether (50 ml) to the residue. Yield: 1.7 g (83%).

N-(2-(10H-phenothiazin-10-yl)ethyl)-N,N-dimethyldodecan-1-aminium bromide (S46):

N,N-Dimethyl-2-(10H-phenothiazin-10-yl)ethan-1-amine (1.0 g; 3.69 mmol) (synthesized by the procedure of M. Blaess, N. Bibak, R. A. Claus, M. Kohl, G. A. Bonaterra, R. Kinscherf, S. Laufer, H.-P. Deigner, European Journal of Medicinal Chemistry 153, 73-104 (2018)), 1-bromododecane (1.0 g; 4.1 mmol), acetonitrile (25 ml). Heated to +50° C. for 5 days. Removed acetonitrile in a nitrogen stream and added diethylether to the residue. Yield: 1.4 g (75%).

TABLE 1

| | | | | | | Molecular |
|---|---|---|---|---|---|---|
| Compound | | | CAS | Molecular | | weight |
| No. | Structure | Chemical name | No. | formula | CLog P* | [g/mol] |
| S1 | 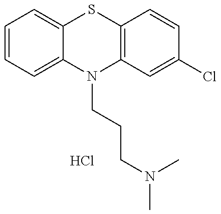 [SMILES: CN(C)CCCN1c2ccccc2Sc2ccc(Cl)cc21 ; Cl] | 3-(2-chloro-10H- phenothiazin-10-yl)- N,N-dimethylpropan- 1-amine hydrochloride | 69-09-0 | C₁₇H₂₀Cl₂N₂S | 5.8447 | 355.32 |
| S2 | 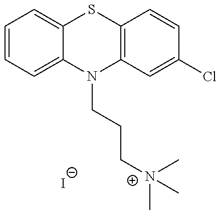 [SMILES: C[N+](C)(C)CCCN1c2ccccc2Sc2ccc(Cl)cc21 ; [I-]] | 3-(2-chloro-10H- phenothiazin-10-yl)- N,N,N- trimethylpropan-1- aminium iodide | 362-02- 7 | C₁₈H₂₂ClIN₂S | 0.9801 | 460.80 |
| S3 | 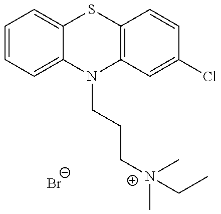 [SMILES: CC[N+](C)(C)CCCN1c2ccccc2Sc2ccc(Cl)cc21 ; [Br-]] | 3-(2-chloro-10H- phenothiazin-10-yl)- N-ethyl-N,N- dimethylpropan-1- aminium bromide | 153871- 17-1 | C₁₉H₂₄BrClIN₂S | 1.5091 | 427.83 |
| S4 | 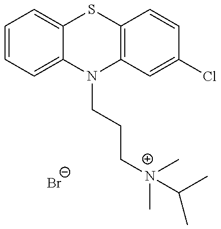 [SMILES: CC(C)[N+](C)(C)CCCN1c2ccccc2Sc2ccc(Cl)cc21 ; [Br-]] | 3-(2-chloro-10H- phenothiazin-10-yl)- N-isopropyl-N,N- dimethypropan-1- aminium bromide | 153871- 19-3 | C₂₀H₂₆BrClN₂S | 1.8181 | 441.86 |
| S5 | 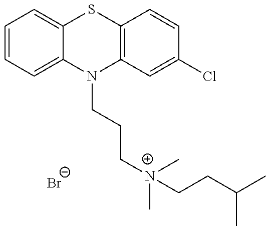 [SMILES: CC(C)CC[N+](C)(C)CCCN1c2ccccc2Sc2ccc(Cl)cc21 ; [Br-]] | N-(3-(2-chloro-10H- phenothiazin-10- yl)propyl)-N,N,3- trimethylbutan-1- aminium bromide | | C₂₂H₃₀BrClN₂S | 2.9661 | 469.91 |
| S6 | 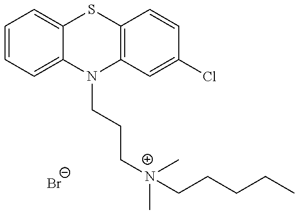 [SMILES: CCCCC[N+](C)(C)CCCN1c2ccccc2Sc2ccc(Cl)cc21 ; [Br-]] | N-(3-(2-chloro-10H- phenothiazin-10- yl)propyl)-N,N- dimethylpentan-1- aminium bromide | | C₂₂H₃₀BrClN₂S | 3.0961 | 469.91 |
| S7 | 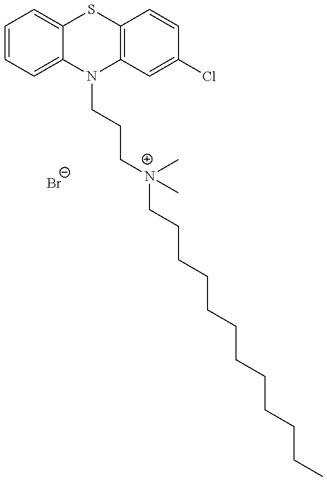 [SMILES: CCCCCCCCCCCC[N+](C)(C)CCCN1c2ccccc2Sc2ccc(Cl)cc21 ; [Br-]] | N-(3-(2-chloro-10H- phenothiazin-10- yl)propyl)-N,N- dimethyldodecan-1- aminium bromide | | C₂₉H₄₄BrClN₂S | 6.7991 | 568.10 |

TABLE 2

| | | | | | | Molecular |
|---|---|---|---|---|---|---|
| Compound | | | CAS | Molecular | | weight |
| No. | Structure | Chemical name | No. | formula | CLog P* | [g/mol] |
| S12 | 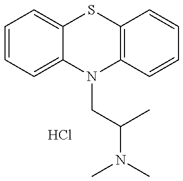 [SMILES: CC(CN1c2ccccc2Sc2ccccc21)N(C)C ; Cl] | N,N-dimethyl-1- (10H-phenothiazin- 10-yl)propan-2- amine hydrochloride | 58-33-3 | C₁₇H₂₁ClN₂S | 4.91242 | 320.88 |
| S13 | 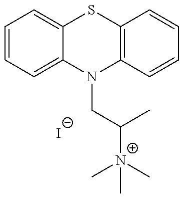 [SMILES: CC(CN1c2ccccc2Sc2ccccc21)[N+](C)(C)C ; [I-]] | N,N,N-trimethyl-1- (10H-phenothiazin- 10-yl)propan-2- aminium iodide | 26212- 80-6 | C₁₈H₂₃IN₂S | 0.4827 | 426.36 |
| S14 | 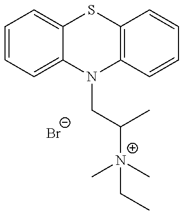 [SMILES: CC[N+](C)(C)C(C)CN1c2ccccc2Sc2ccccc21 ; [Br-]] | N-ethyl-N,N- dimethyl-1-(10H- phenothiazin-10- yl)propan-2-aminium bromide | 3366- 74-3 | C₁₉H₂₅BrN₂S | 1.0117 | 393.39 |
| S15 | 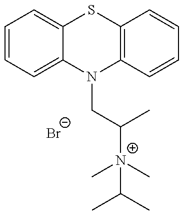 [SMILES: CC(C)[N+](C)(C)C(C)CN1c2ccccc2Sc2ccccc21 ; [Br-]] | N-isopropyl-N,N- dimethyl-1-(10H- phenothiazin-10- yl)propan-2-aminium bromide | | C₂₀H₂₇BrN₂S | 1.3207 | 407.41 |
| S16 | 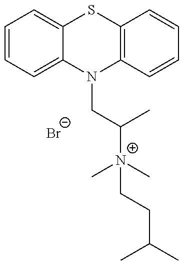 [SMILES: CC(C)CC[N+](C)(C)C(C)CN1c2ccccc2Sc2ccccc21 ; [Br-]] | N-(1-(10H- phenothiazin-10- yl)propan-2-yl)- N,N,3- trimethylbutan-1- aminium bromide | 3366- 78-7 | C₂₂H₃₁BrN₂S | 2.4687 | 435.47 |
| S17 | 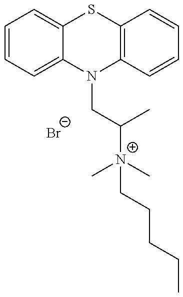 [SMILES: CCCCC[N+](C)(C)C(C)CN1c2ccccc2Sc2ccccc21 ; [Br-]] | N-(1-(10H- phenothiazin-10- yl)propan-2-yl)-N,N- dimethylpentan-1- aminium bromide | 3366- 77-6 | C₂₂H₃₁BrN₂S | 2.5987 | 435.47 |
| 18 | 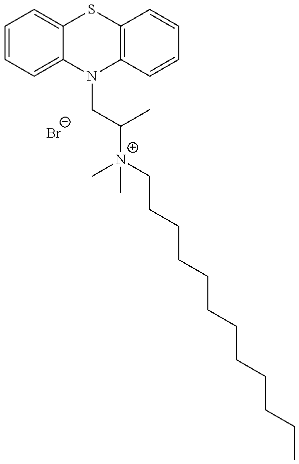 [SMILES: CCCCCCCCCCCC[N+](C)(C)C(C)CN1c2ccccc2Sc2ccccc21 ; [Br-]] | N-(1-(10H- phenothiazin-10- yl)propan-2-yl)-N,N- dimethyldodecan-1- aminium bromide | | C₂₉H₄₅BrN₂S | 6.3017 | 533.66 |

TABLE 3

| Com- | | | | | | Molecular |
|---|---|---|---|---|---|---|
| pound | | | | Molecular | | weight |
| No. | Structure | Chemical name | CAS No. | formula | CLog P* | [g/mol] |
| S23 | 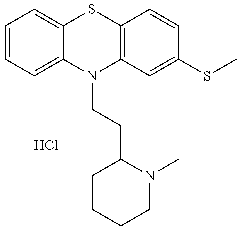 [SMILES: CSc1ccc2c(c1)N(CCC1CCCCN1C)c1ccccc1S2 ; Cl] | 10-(2-(1- methylpiperidin-2- yl)ethyl)-2- (methylthio)-10H- phenothiazine hydrochloride | 130-61-0 | C₂₁H₂₇ClN₂S₂ | 6.734 | 407.03 |
| S24 | 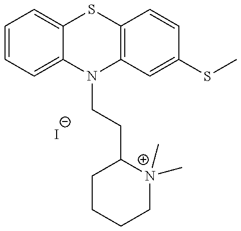 [SMILES: CSc1ccc2c(c1)N(CCC1CCCC[N+]1(C)C)c1ccccc1S2 ; [I-]] | 1,1-dimethyl-2-(2- (2-(methylthio)- 10H-phenothiazin- 10- yl)ethyl)piperidin- 1-ium iodide | 74167-56- 9 | C₂₂H₂₉IN₂S₂ | 2.552 | 512.51 |
| S25 | 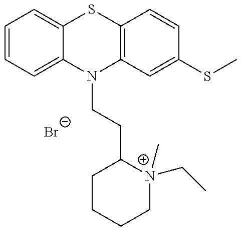 [SMILES: CC[N+]1(C)CCCCC1CCN1c2ccccc2Sc2ccc(SC)cc21 ; [Br-]] | 1-ethyl-1-methyl-2- (2-(2-(methylthio)- 10H-phenothiazin- 10- yl)ethyl)piperidin- 1-ium bromide | | C₂₃H₃₁BrN₂S₂ | 3.081 | 479.54 |
| S26 | 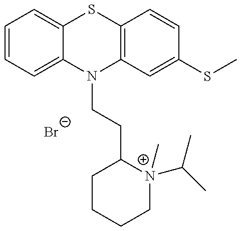 [SMILES: CSc1ccc2c(c1)N(CCC1CCCC[N+]1(C)C(C)C)c1ccccc1S2 ; [Br-]] | 1-isopropyl-1- methyl-2-(2-(2- (methylthio)-10H- phenothiazin-10- yl)ethyl)piperidin- 1-ium bromide | | C₂₄H₃₃BrN₂S₂ | 3.39 | 493.57 |
| S27 | 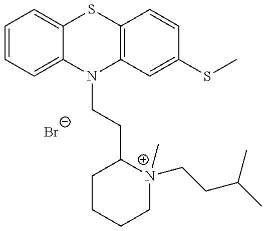 [SMILES: CSc1ccc2c(c1)N(CCC1CCCC[N+]1(C)CCC(C)C)c1ccccc1S2 ; [Br-]] | 1-isopentyl-1- methyl-2-(2-(2- (methylthio)-10H- phenothiazin-10- yl)ethyl)piperidin- 1-ium bromide | | C₂₆H₃₇BrN₂S₂ | 4.538 | 521.62 |
| S28 | 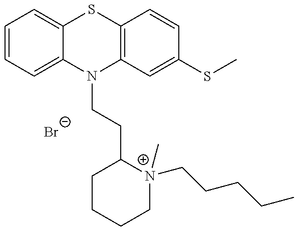 [SMILES: CCCCC[N+]1(C)CCCCC1CCN1c2ccccc2Sc2ccc(SC)cc21 ; [Br-]] | 1-methyl-2-(2-(2- (methylthio)-10H- phenothiazin-10- yl)ethyl)-1- pentylpiperidin-1- ium bromide | | C₂₈H₃₇₈rN₂S₂ | 4.668 | 521.62 |
| S29 | 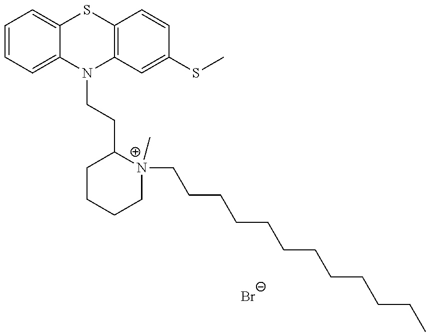 [SMILES: CCCCCCCCCCCC[N+]1(C)CCCCC1CCN1c2ccccc2Sc2ccc(SC)cc21 ; [Br-]] | 1-dodecyl-1- methyl-2-(2-(2- (methylthio)-10H- phenothiazin-10- yl)ethyl)piperidin- 1-ium bromide | | C₃₃H₅₁BrN₂S₂ | 8.371 | 619.81 |

*(estimated by ChemBioDraw Ultra 14.0 - does not include counterion)

TABLE 4

| | | | | | | Molecular |
|---|---|---|---|---|---|---|
| Compound | | | | Molecular | | weight |
| No. | Structure | Chemical name | CAS No. | formula | CLog P* | [g/mol] |
| S34 | 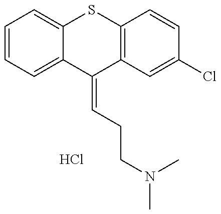 [SMILES: CN(C)CC/C=C1/c2ccccc2Sc2ccc(Cl)cc21 ; Cl] | (Z)-3-(2-chloro-9H- thioxanthen-9- ylidene)-N,N- dimethylpropan-1- amine hydrochloride | 6469-93-8 | C₁₈H₁₉Cl₂NS | 5.888 | 352.32 |
| S35 | 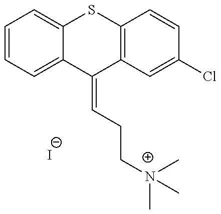 [SMILES: C[N+](C)(C)CC/C=C1/c2ccccc2Sc2ccc(Cl)cc21 ; [I-]] | (Z)-3-(2-chloro-9H- thioxanthen-9- ylidene)-N,N,N- trimethylpropan-1- aminium iodide | 6469-99-4 | C₁₉H₂₁ClINS | 0.208 | 457.80 |
| S36 | 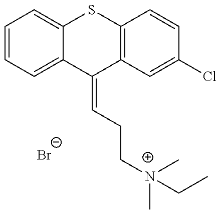 [SMILES: CC[N+](C)(C)CC/C=C1/c2ccccc2Sc2ccc(Cl)cc21 ; [Br-]] | (Z)-3-(2-chloro-9H- thioxanthen-9- ylidene)-N-ethyl- N,N- dimethylpropan-1- aminium bromide | 1039634- 27-9 | C₂₀H₂₃BrClNS | 0.737 | 424.83 |
| S37 | 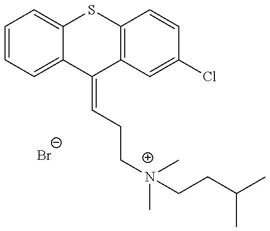 [SMILES: CC(C)CC[N+](C)(C)CC/C=C1/c2ccccc2Sc2ccc(Cl)cc21 ; [Br-]] | (Z)-N-(3-(2-chloro- 9H-thioxanthen-9- ylidene)propyl)- N,N,3- trimethylbutan-1- aminium bromide | | C₂₃H₂₉BrClNS | 2.194 | 466.91 |
| S38 | 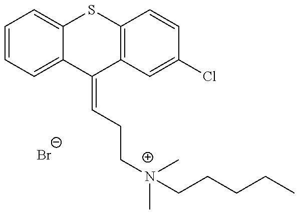 [SMILES: CCCCC[N+](C)(C)CC/C=C1/c2ccccc2Sc2ccc(Cl)cc21 ; [Br-]] | (Z)-N-(3-(2-chloro- 9H-thioxanthen-9- ylidene)propyl)- N,N- dimethypentan-1- aminium bromide | | C₂₃H₂₉BrClNS | 2.324 | 466.91 |
| S39 | 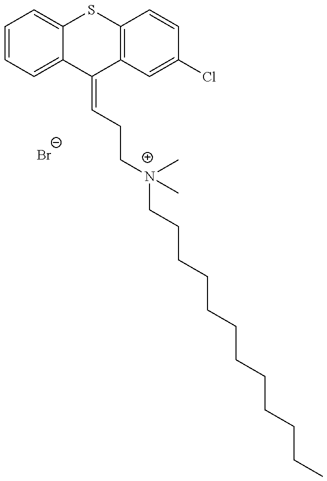 [SMILES: CCCCCCCCCCCC[N+](C)(C)CC/C=C1/c2ccccc2Sc2ccc(Cl)cc21 ; [Br-]] | (Z)-N-(3-(2-chloro- 9H-thioxanthen-9- ylidene)propyl)- N,N- dimethyldodecan- 1-aminium bromide | | C₃₀H₄₃BrClNS | 6.027 | 565.10 |

TABLE 5

| | | | | | | Molecu- |
|---|---|---|---|---|---|---|
| Com- | | | | | | lar |
| pound | | | CAS | Molecular | | weight |
| No. | Structure | Chemical name | No. | formula | CLog P* | [g/mol] |
| S40 | 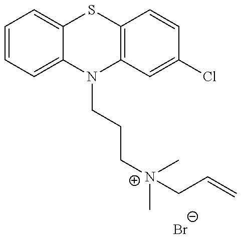 [SMILES: C=CC[N+](C)(C)CCCN1c2ccccc2Sc2ccc(Cl)cc21 ; [Br-]] | N-(3-(2-chloro- 10H-phenothiazin- 10-yl)propyl)- N,N-dimethyl- prop-2-en- 1-aminium bromide | | C₂₀H₂₄BrClN₂S | 1.7541 | 439.84 |
| S41 | 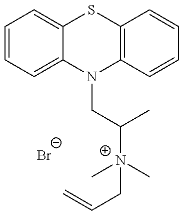 [SMILES: C=CC[N+](C)(C)C(C)CN1c2ccccc2Sc2ccccc21 ; [Br-]] | N-(1-(10H- phenothiazin-10- yl)propan-2-yl)- N,N-dimethyl- prop-2-en-1- aminium bromide | | C₂₀H₂₅BrN₂S | 2.1573 | 405.40 |
| S42 | 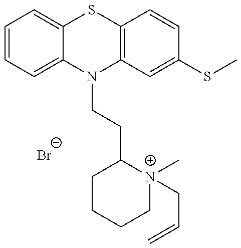 [SMILES: C=CC[N+]1(C)CCCCC1CCN1c2ccccc2Sc2ccc(SC)cc21 ; [Br-]] | 1-allyl-1-methyl- 2-(2-(2- (methylthio)- 10H-pheno- thiazin-10- yl)ethyl)piperidin- 1-ium bromide | | C₂₄H₃₁BrN₂S₂ | 3.326 | 491.55 |
| S43 | 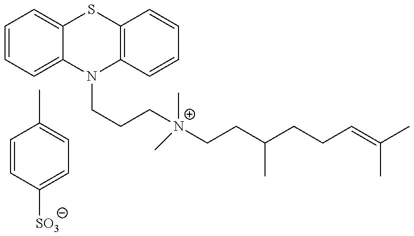 | N-(3-(10H- phenothiazin-10- yl)propyl)- N,N,3,7- tetramethyloct-6- en-1-aminium 4- methylbenzene- sulfonate | | C₃₄H₄₆N2O₃S₂ | 4.0965 | 594.87 |
| S44 | 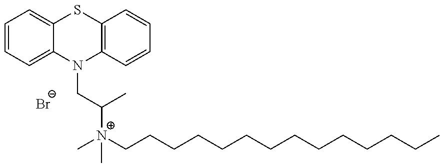 [SMILES: CCCCCCCCCCCCCC[N+](C)(C)C(C)CN1c2ccccc2Sc2ccccc21 ; [Br-]] | N-(1-(10H- phenothiazin-10- yl)propan-2-yl)- N,N-dimethyl- tetradecan- 1-aminium bromide | | C₃₁H₄₉BrN₂S | 7.3597 | 561.71 |
| S45 | 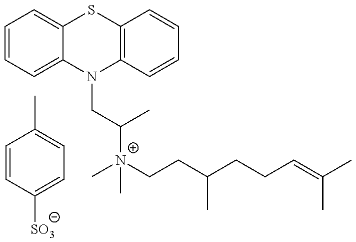 | N-(1-(10H- phenothiazin-10- yl)propan-2-yl)- N,N,3,7- tetramethyloct-6- en-1-aminium 4- methylbenzene- sulfonate | | C₃₄H₄₆N2O₃S₂ | 4.4997 | 594.87 |
| S46 | 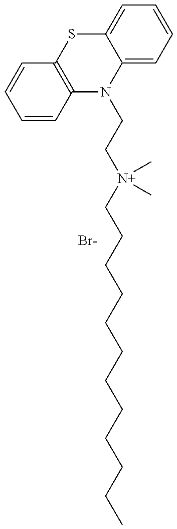 [SMILES: CCCCCCCCCCCC[N+](C)(C)CCN1c2ccccc2Sc2ccccc21 ; [Br-]] | N-(2-(10H- phenothiazin-10- yl)ethyl)-N,N- dimethyldodecan- 1-aminium bromide | | C₂₈H₄₃BrN₂S | 5.9927 | 519.63 |

### Example 3

The aim of the study was to determine the Minimal Inhibitory Concentration (MIC) of four original compounds (thioridazine hydrochlorid, promethazine hydrochlorid, chlorpromazine hydrochlorid and chlorprothixene hydrochlorid) and 36 derivatives hereof, for ten different bacterial strains.

Ten different bacterial strains, representing 8 different bacteria species, were included in the strain collection. For all strains, a MIC value for each compound was established.

TABLE 6
Bacterial strains for which MIC values were determined for all 38 compound.

| | | Strain | | | |
|---|---|---|---|---|---|
| Genus | Species | name | Gram | Ref. | Comments |
| Staphylococcus | aureus | JE2 | + | [1] | MRSA |
| | | | | | strain |
| Staphylococcus | aureus | CC398 | + | | MRSA |
| | | | | | strain |
| Bacillus | cereus | ATCC_11778 | + | America-type | |
| | | | | culture | |
| | | | | collection | |
| Enterococcus | faecium | Un-named | + | | |
| Enterococcus | faecalis | Un-named | + | | |
| Staphylococcus | pseudintermedius | DK729 | + | | |
| Streptocococcus | equi | JTR204 | + | | |
| Listeria | monocytogenes | Un-named | + | | |
| Escherichia | coli | APEC_O2 | − | [2] | |
| Salmonella | enteritidis | Fagtype 6 | − | | |

[1] Baek K T, Thogersen L, Mogenssen R G, Mellergaard M, Thomsen L E, Petersen A, Skov S, Cameron D R, Peleg A Y, Frees D. Stepwise decrease in daptomycin susceptibility in clinical Staphylococcus aureus isolates asso ciated with an initial mutation in rpoB and a compensatory inactivation of the clpX gene. Antimicrob Agents Chemother. 2015; 59: 6983-6991.
[2] Jorgensen S L, Kudirkiene E, Li L, Christensen J P, Olsen J E, Nolan L, Olsen R H. Chromosomal features of Escherichia coli serotype O2:K2, an avian pathogenic E. coli. Stand Genomic Sci. 2017; 12: 33.

TABLE 7
Bacterial strains for which MIC values were determined
for chlorpromazine hydrochloride and dodecyl.

| | | Strain | | | |
|---|---|---|---|---|---|
| Genus | Species | name | Gram | Ref. | Comments |
| Escherichia | coli | ESBL1 | − | [3] | Tetracycline |
| | | | | | resistant |
| Escherichia | coli | ESBL10 | − | [3] | Tetracycline |
| | | | | | resistant |
| Escherichia | coli | ESBL19 | − | [3] | Tetracycline |
| | | | | | resistant |
| Escherichia | coli | ESBL28 | − | [3] | Tetracycline |
| | | | | | resistant |
| Escherichia | coli | ESBL37 | − | [3] | Tetracycline |
| | | | | | resistant |
| Escherichia | coli | ESBL46 | − | [3] | Tetracycline |
| | | | | | resistant |
| Escherichia | coli | E2 | − | [4] | ESBL¹ |
| Escherichia | coli | E4 | − | [4] | ESBL |
| Escherichia | coli | E5 | − | [4] | ESBL |
| Escherichia | coli | E6 | − | [4] | ESBL |
| Escherichia | coli | E24 | − | [4] | ESBL |
| Escherichia | coli | E38 | − | [4] | ESBL |

¹Extended spectrum beta-lactamase
[3] Olsen R H, Bisgaard M, Lohren U, Robineau B, Christensen H. Extended-spectrum beta-lactamase-producing Escherichia coli isolated from poultry: a review of current problems, illustrated with some laboratory findings. Avian Pathol. 2014; 43: 199-208.
[4] Ahmad A, Zachariasen C, Christiansen L E, Graesboll K, Toft N, Matthews L, Damborg P, Agerso Y, Olsen J E, Nielsen S S. Pharmacodynamic modelling of in vitro activity of tetracycline against a representative, naturally occurring population of porcine Escherichia coli. Acta Vet Scand. 2015; 57: 79.

TABLE 7b
Bacterial strains for which MIC and MBC
values were determined for S18 and S43.

| | | Strain | | |
|---|---|---|---|---|
| Genus | Species | name | Gram | Ref |
| Cutibacterium | acnes | X10 | + | Clinical, |
| | | | | multiresistent |
| | | | | isolate |
| Staphylococcus | Epidermidis | RP62A | + | Clinical, |
| | | | | multiresistent |
| | | | | isolate |
| Campylobacter | jejuni | B31C6 | − | Clinical |
| | | | | isolate |

Method:

The MIC for each compound for each bacteria strain was determined by the serial dilution method following the CLSI guidelines (CLSI Clinical and Laboratory Standards Institute: Performance Standards for Antimicrobial Susceptibility Testing: Twenty-First Informational Supplement M100-S25. Wayne, Pa., USA; 2015.) All compounds were diluted in autoclaved H₂O and stored at 4° C. For each compound, the concentration tested ranged from 0.0125 to 256 mg/L by two-fold increase. Bacterial inoculums were prepared by inoculating 9 ml of H₂O with bacterial colonies from agar plate (Oxoid, Roskilde Denmark) supplemented with bovine blood to a final yield a final density of 10⁸ colony forming unit (CFU)/mL using a Sensititre™ Nephelometer (Thermo Scientific, Roskilde, Denmark). These inoculums were each in diluted 1:100 in Müller Hinton (MH) broth (Sigma, Copenhagen, Denmark). From the diluted inoculums 100 μl was transferred to each well in a 96-well plate and mixed with the compound to be MIC determined. The plate was incubated for 24 hours at 37° C. (without shaking). The MIC value was determined as the lowest concentration in which no bacterial growth could be visually detected. All MIC determinations were done in duplicates. If growth was observed in the wells contained 256 mg/L, the MIC value was set to 256 mg/L although the true value may be significantly higher. Since all derivatives are new, no control strains (with reference MIC values) were available.

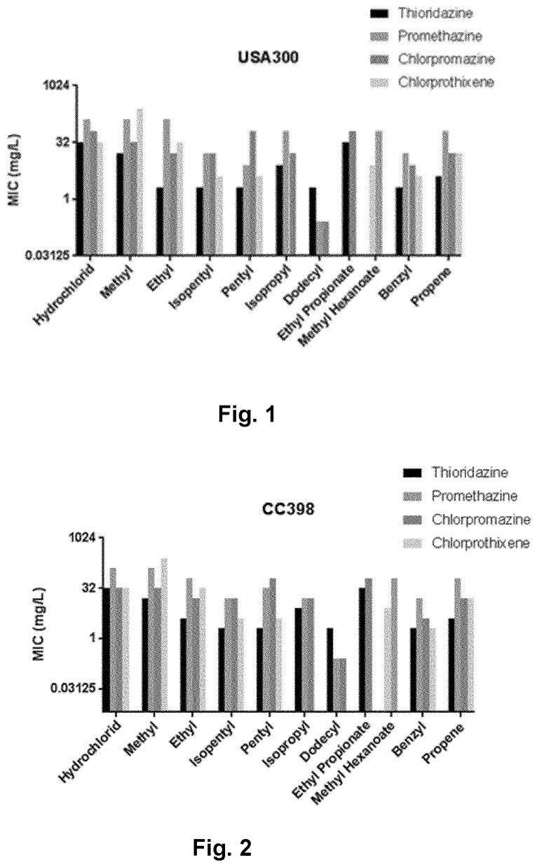

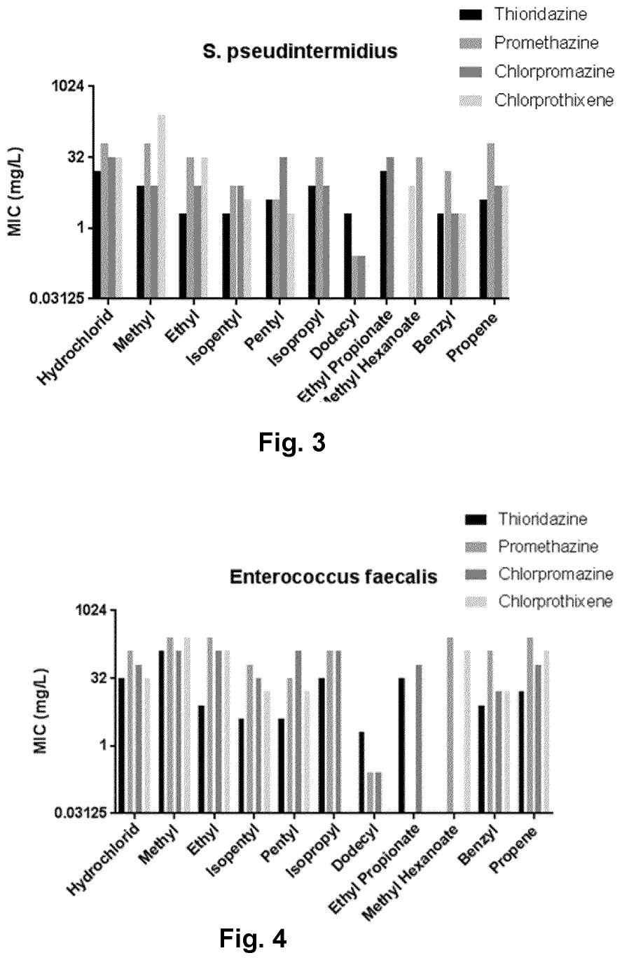

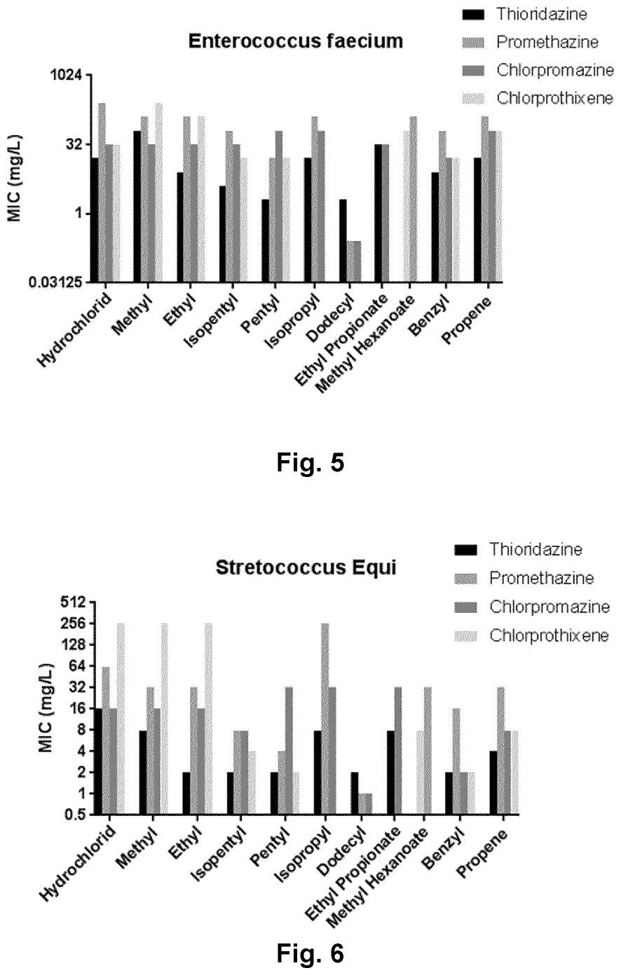

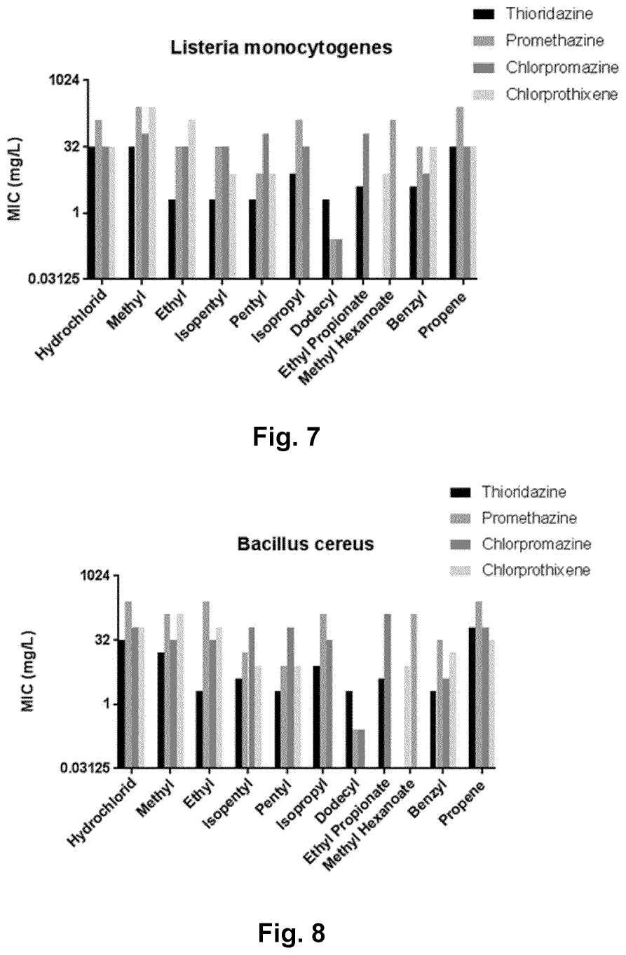

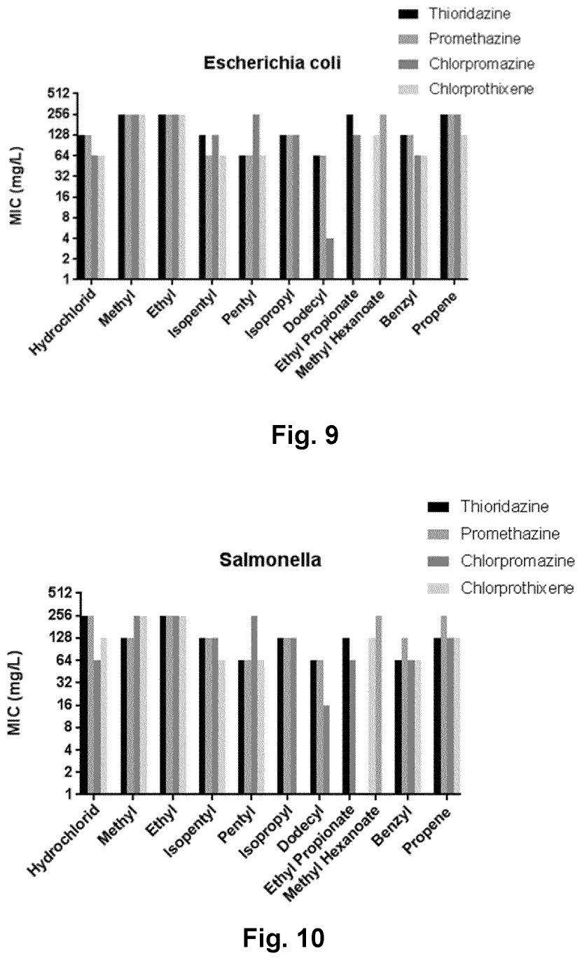

The results are presented in table 8, 9 and 10 and in FIGS. 1 to 10.

TABLE 8

| | | | |
|---|---|---|---|
| Thioridazine | Promethazine | Chlorpromazine | Chlorprothixene |

S. aureus USA300

| | | | | |
|---|---|---|---|---|
| Hydrochloride | 32 | 128 | 64 | 32 |
| Methyl | 16 | 128 | 32 | 256 |
| Ethyl | 2 | 128 | 16 | 32 |
| 1-Isopentyl | 2 | 16 | 16 | 4 |
| 1-Pentyl | 2 | 8 | 64 | 4 |
| Isopropyl | 8 | 64 | 16 | |
| 1-Dodecyl | 4 | 0.5 | 0.5 | 4 |
| 3-Ethoxy-3- | 32 | | 64 | |
| oxo-1-propyl | | | | |
| 6-Methoxy-6- | | 64 | | 8 |
| oxo-1-hexyl | | | | |
| Benzyl | 2 | 16 | 8 | 4 |
| 2-Propene-1-yl | 4 | 64 | 16 | 16 |
| 3,7-dimethyloct- | | 4 | | |
| 6-en-1-yl | | | | |
| 1-Tetradecyl | | 8/16 | | |
| 3,7-dimethyloct- | | | 2 (Promazine) | |
| 6-en-1-yl | | | | |

E. faecalis

| | | | | |
|---|---|---|---|---|
| Hydrochloride | 32 | 128 | 64 | 32 |
| Methyl | 128 | 256 | 128 | 256 |
| Ethyl | 8 | 256 | 128 | 128 |
| 1-Isopentyl | 4 | 64 | 32 | 16 |
| 1-Pentyl | 4 | 32 | 128 | 16 |
| Isopropyl | 32 | 128 | 128 | |
| 1-Dodecyl | 4 | 0.5 | 0.5 | |
| 3-Ethoxy-3- | 32 | | 64 | |
| oxo-1-propyl | | | | |
| 6-Methoxyy-6- | | 256 | | 128 |
| oxo-1-hexyl | | | | |
| Benzyl | 8 | 128 | 16 | 16 |
| 2-Propene-1-yl | 16 | 256 | 64 | 128 |

E. coli APEC O2

| | | | | |
|---|---|---|---|---|
| Hydrochloride | 128 | 128 | 64 | 64 |
| Methyl | 256 | 256 | 256 | 256 |
| Ethyl | 256 | 256 | 256 | 256 |
| 1-Isopentyl | 128 | 64 | 128 | 64 |
| 1-Pentyl | 64 | 64 | 256 | 64 |
| Isopropyl | 128 | 128 | 128 | |
| 1-Dodecyl | 64 | 64 | 4 | |
| 3-Ethoxy-3- | 256 | | 128 | |
| oxo-1-propyl | | | | |
| 6-Methoxy-6- | | 256 | | 128 |
| oxo-1-hexyl | | | | |
| Benzyl | 128 | 128 | 64 | 64 |
| 2-Propene-1-yl | 256 | 256 | 256 | 128 |

Staphylococus pseudintermedius

| | | | | |
|---|---|---|---|---|
| Hydrochloride | 16 | 64 | 32 | 32 |
| Methyl | 8 | 64 | 8 | 256 |
| Ethyl | 2 | 32 | 8 | 32 |
| 1-Isopentyl | 2 | 8 | 8 | 4 |
| 1-Pentyl | 4 | 4 | 32 | 2 |
| Isopropyl | 8 | 32 | 8 | |
| 1-Dodecyl | 2 | 0.25 | 0.25 | |
| 3-Ethoxy-3- | 16 | | 32 | |
| oxo-1-propyl | | | | |
| 6-Methoxy-6- | | 32 | | 8 |
| oxo-1-hexyl | | | | |
| Benzyl | 2 | 16 | 2 | 2 |
| 2-Propene-1-yl | 4 | 64 | 8 | 8 |

Streptococcus equi

| | | | | |
|---|---|---|---|---|
| Hydrochloride | 16 | 64 | 16 | 256 |
| Methyl | 8 | 32 | 16 | 256 |
| Ethyl | 2 | 32 | 16 | 256 |
| 1-Isopentyl | 2 | 8 | 8 | 4 |
| 1-Pentyl | 2 | 4 | 32 | 2 |
| Isopropyl | 8 | 256 | 32 | |
| 1-Dodecyl | 2 | 1 | 1 | |
| 3-Ethoxy-3- | 8 | | 32 | |
| oxo-1-propyl | | | | |
| 6-Methoxy-6- | | 32 | | 8 |
| oxo-1-hexyl | | | | |
| Benzyl | 2 | 16 | 2 | 2 |
| 2-Propene-1-yl | 4 | 32 | 8 | 8 |

S. aureus CC398

| | | | | |
|---|---|---|---|---|
| Hydrochloride | 32 | 128 | 32 | 32 |
| Methyl | 16 | 128 | 32 | 256 |
| Ethyl | 4 | 64 | 16 | 32 |
| Isopentyl | 2 | 16 | 16 | 4 |
| Pentyl | 2 | 32 | 64 | 4 |
| Isopropyl | 8 | 16 | 16 | |
| Dodecyl | 2 | 0.25 | 0.25 | |
| 3-Ethoxy-3- | 32 | | 64 | |
| oxo-1-propyl | | | | |
| 6-Methoxyyl-6- | | 64 | | 8 |
| oxo-1-hexyl | | | | |
| Benzyl | 2 | 16 | 4 | 2 |
| 2-Propene-1-yl | 4 | 64 | 16 | 16 |

E. faecium

| | | | | |
|---|---|---|---|---|
| Hydrochloride | 16 | 256 | 32 | 32 |
| Methyl | 64 | 128 | 32 | 256 |
| Ethyl | 8 | 128 | 32 | 128 |
| Isopentyl | 4 | 64 | 32 | 16 |
| Pentyl | 2 | 16 | 64 | 16 |
| Isopropyl | 16 | 128 | 64 | |
| Dodecyl | 2 | 0.25 | 0.25 | |
| 3-Ethoxy-3- | 32 | | 32 | |
| oxo-1-propyl | | | | |
| 6-Methoxyyl-6- | | 128 | | 64 |
| oxo-1-hexyl | | | | |
| Benzyl | 8 | 64 | 16 | 16 |
| 2-Propene-1-yl | 16 | 128 | 64 | 64 |

Salmonella enteritidis

| | | | | |
|---|---|---|---|---|
| Hydrochloride | 256 | 256 | 64 | 128 |
| Methyl | 128 | 128 | 256 | 256 |
| Ethyl | 256 | 256 | 256 | 256 |
| Isopentyl | 128 | 128 | 128 | 64 |
| Pentyl | 64 | 64 | 256 | 64 |
| Isopropyl | 128 | 128 | 128 | |
| Dodecyl | 64 | 64 | 16 | |
| 3-Ethoxy-3- | 128 | | 64 | |
| oxo-1-propyl | | | | |
| 6-Methoxyyl-6- | | 256 | | 128 |
| oxo-1-hexyl | | | | |
| Benzyl | 64 | 128 | 64 | 64 |
| 2-Propene-1-yl | 128 | 256 | 128 | 128 |

Listeria monocytogenes

| | | | | |
|---|---|---|---|---|
| Hydrochloride | 32 | 128 | 32 | 32 |
| Methyl | 32 | 256 | 64 | 256 |
| Ethyl | 2 | 32 | 32 | 128 |
| Isopentyl | 2 | 32 | 32 | 8 |
| Pentyl | 2 | 8 | 64 | 8 |
| Isopropyl | 8 | 128 | 32 | |
| Dodecyl | 2 | 0.25 | 0.25 | |
| 3-Ethoxy-3- | 4 | | 64 | |
| oxo-1-propyl | | | | |
| 6-Methoxyyl-6- | | 128 | | 8 |
| oxo-1-hexyl | | | | |
| Benzyl | 4 | 32 | 8 | 32 |
| 2-Propene-1-yl | 32 | 256 | 32 | 32 |

Bacillus cereus

| | | | | |
|---|---|---|---|---|
| Hydrochloride | 32 | 256 | 64 | 64 |
| Methyl | 16 | 128 | 32 | 128 |
| Ethyl | 2 | 256 | 32 | 64 |
| Isopentyl | 4 | 16 | 64 | 8 |
| Pentyl | 2 | 8 | 64 | 8 |
| Isopropyl | 8 | 128 | 32 | |
| Dodecyl | 2 | 0.25 | 0.25 | |
| 3-Ethoxy-3- | 4 | | 128 | |
| oxo-1-propyl | | | | |
| Metyl hexanoate | | 128 | | 8 |
| Benzyl | 2 | 32 | 4 | 16 |
| 2-Propene-1-yl | 64 | 256 | 64 | 32 |

TABLE 9

| | |
|---|---|
| Strain | Chlorpromazine |

| name | Hydrochl | Dodecyl |
|---|---|---|
| ESBL1 | 32/64 | 4/4 |
| ESBL10 | 64/64 | 8/8 |
| ESBL19 | 32/32 | 4/4 |
| ESBL28 | 32/32 | 4/4 |
| ESBL37 | 32/32 | 4/4 |
| ESBL46 | 32/32 | 4/8 |
| E2 | 64/32 | 8/4 |
| E4 | 64/64 | 4/4 |
| E5 | 32/32 | 4/4 |
| E6 | 32/32 | 4/4 |
| E24 | 64/32 | 4/4 |
| E38 | 64/32 | 4/4 |

TABLE 10

| | N-(3-(10H- | | | | |
|---|---|---|---|---|---|
| | phenothiazin- | | | | |
| | 10-yl)propyl)- | | | | |
| | N,N,3,7- | | | | |
| | tetramethyloct- | | | | |
| | 6-en-1-aminium 4- | | Promethazine | | |
| | ethylbenzenesulfonate | | dodecyl | | Original |
| strain | (S43) mg/L | | (S18) mg/L | | of strain |
| Staphylococcus | 2 | (MIC) | 0.25 | (MIC) | Multiresistant |
| epidermidis | 2 | (MBC) | 0.25 | (MBC) | clinical |
| | | | | | isolate |
| Campylobacter | 1 | (MIC) | Not | | Clinical |
| jejuni | 1 | (MBC) | evaluated | | isolate |
| Cutibacterium | 1 | (MIC) | 0.5 | (MIC) | Multiresistant |
| acnes | 1 | (MBC) | 0.5 | (MBC) | clinical |
| | | | | | isolate |

### Example 4

To assess the permeability of the compounds, the apical to basolateral flux/permeability of different experimental compounds across monolayers of IPEC-J2 MDR1 cells seeded and cultured on Transwell supports was investigated.

Materials and Methods:

Materials

Bovine serum albumin (BSA), Hank's balanced salt solution (HBSS), Fetal bovine serum (FBS), 2-[4-(2-hydroxyethyl) piperazin-1-yl] ethanesulfonic acid (HEPES), Ultima Gold Scintillation Fluid, Transwell® Permeable supports (1.13 cm2, 0.4 μm pore size), Cytotoxicity Detection Kit (LDH).

Experimental Compounds
Compound 1: 10-(2-(1-methylpiperidin-2-yl)ethyl)-2-(methylthio)-10H-phenothiazine hydrochloride (S23);
Compound 2: 1-ethyl-1-methyl-2-(2-(2-(methylthio)-1-OH-phenothiazin-10-yl)ethyl)piperidin-1-ium bromide (S25);
Compound 3: 1-isopentyl-1-methyl-2-(2-(2-(methylthio)-10H-phenothiazin-10-yl)ethyl)piperidin-1-ium bromide (S27);
Compound 4: 1-methyl-2-(2-(2-(methylthio)-10H-phenothiazin-10-yl)ethyl)-1-pentylpiperidin-1-ium bromide (S28).

The compounds were dissolved in ultra pure water (MilliQ) to a concentration of 1 mM and further diluted in HBSS supplemented with 0.05% BSA and 10 mM HEPES, pH 7.4 to a final concentration of 10 μM.

For transport experiments, IPEC-J2 cells were seeded onto to permeable supports (T12, Corning cci-3401) and cultured for 15-17 days. Culturemedium was changed every other day in both the apical and basolateral chamber. On the day of transport, the cells were equilibrated to ambient temperatures and the transepithelial electrical resistance (TEER) was measured across the cell monolayers. Subsequently, the cell layers were washed twice with HBSS and the TEER was measured again. The transport experiment was started by replacing the HBSS in the apical chamber with HBSS solutions of the four experimental compounds. Over a period of two hours samples of 100 μL where taken from the basolateral chamber at t=15, 30, 45, 60, 90 and 120 minutes. All samples were stored at −20° C. until analysis by means of HPLC-MS. All four compounds were tested in triplicate. After the transport study was stopped, the cell layers were washed twice with HBSS and the TEER was measured.

In a parallel transport experiment, ¹⁴C-mannitol (0.5 μCi/mL) was added to all four compound solutions. The experiment was performed as described above, with the exceptions that the four compounds were tested in duplicate and that samples were mixed with 2 mL of Ultima Gold Scintillation Fluid and analysed by liquid scintillation counting.

Lactate Dehydrogenase (LDH) assay

LDH-release from cell monolayers was measured in samples of the donor solutions taken from the apical compartment after 120 minutes of exposure on IPEC-J2 MDR1 cell monolayers. Cell lysate of cells treated with ultrasound was added to the analysis as a positive control. Samples were analysed according to manufacturer's protocol in duplicate.

Quantification of Experimental Compounds

Lc-Specific:

Column: Unknown. Phenomenex EVO C18. Column temp: 40° C. Mobilphases: A: MilliQ+0.1% Formic acid; B: Acetonitrile+0.1% Formic acid. Flow: 0.5 mL/min. Injection: 5 uL. UV Detection: 254 nm.

Gradient:

| Time (minutes) | Solvent B (%) |
|---|---|
| 0 | 20 |
| 0.5 | 20 |
| 2 | 40 |
| 8 | 65 |
| 14 | 20 |

Runtime 15 min.

MS-specific:

Ionization Mode: MM-ESI. Polarity: Positive. Spray Chamber having Gas temp of 250° C., Vaporizer of 200° C., Drying gas of 12 L/min and Neb. pres. at 35 psig. VCap: 4000 V.

Corona: 4 A.

| SIM Ion | Fragmentor |
|---|---|
| 371.2 70 | Compound 1 |
| 399.2 70 | Compound 2 |
| 441.3 70 | Compound 3 |
| 441.2 70 | Compound 4 |

Results:

Apical to Basolateral Transport Across IPEC-J2 MDR1 Cells.

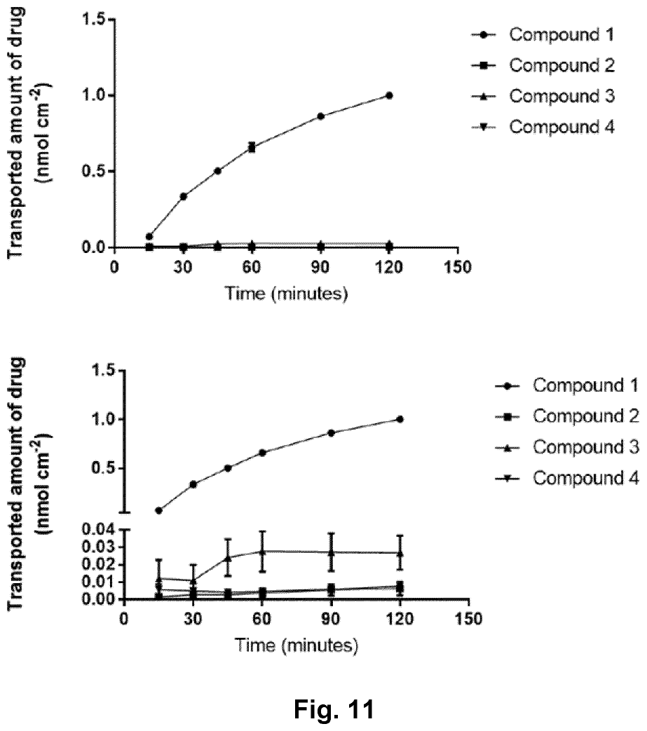

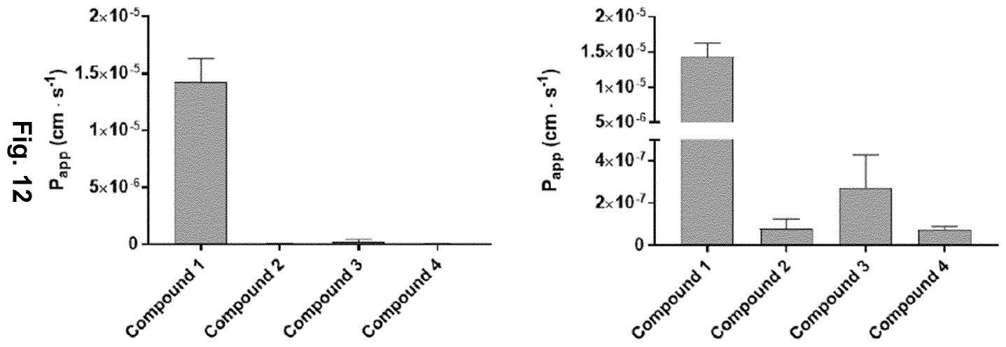

FIG. 11 shows the flux curves of the four compounds. The highest transport (flux) was observed for Compound 1, which is a reference compound. The flux of the other three compounds (Compound 2, 3, and 4) was by comparison much lower than the flux observed for Compound 1. The appearance of Compound 2, 3 and 4 in the receptor chamber was barely detectable with the analytical method employed in this project. The logarithmic-like shape of the flux curve for Compound 1 indicates that the flux of this compound was changing during the transport experiments (lack of steady-state flux), and as a consequence, it was difficult to calculate an accurate permeability. Permeability calculations were therefore done on smaller sections of the flux curves (at least three data points) rather than using all data points.

FIG. 12 shows the calculated permeabilities for the four compounds. However, due to the lack of steady-state flux, the calculated permeabilities should be evaluated with caution. The calculated permeabilities show that the transport of Compound 1 was at least 50-fold higher than Compound 3 and almost 200-fold higher than Compound 2 and 4.

Mannitol Transport

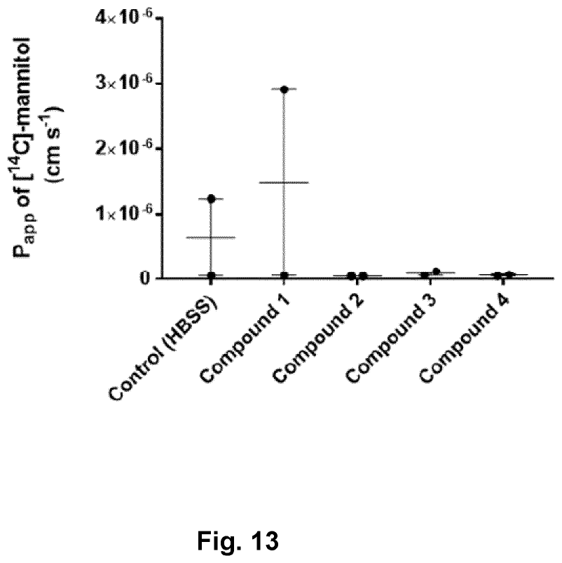

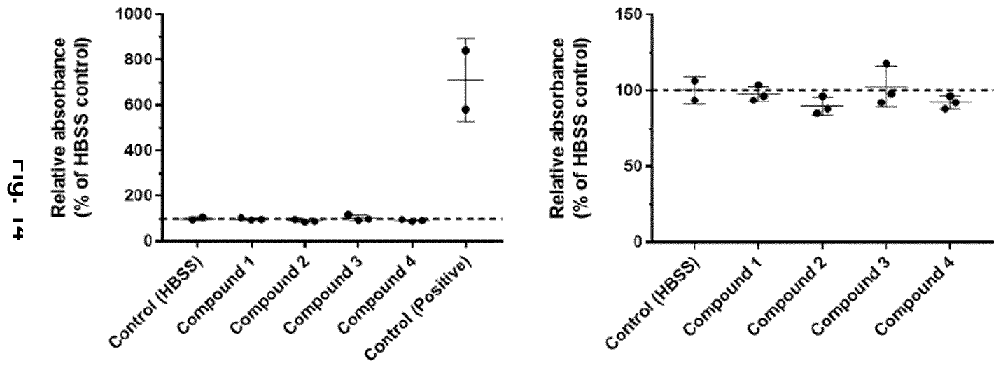

FIG. 13 shows the ¹⁴C-mannitol transport across IPEC-J2 MDR1 cells exposed to either supplemented HBSS (control) or 10 μM solutions of the four compounds. The expected mannitol permeability across an unaffected monolayer of IPEC-J2 MDR1 cells is below 1×10−6 cm/s. Most of the values obtained in the present study were below this threshold, however one replicate for cells exposed to supplemented HBSS and one replicate for cells exposed to 10 μM Compound 1 was above. The immediate conclusion to the observed mannitol transport data would be that the barrier properties of the monolayers, from which the increased mannitol transport was observed, were compromised during the experiment. However, since only replicate of the two treatments showed increased mannitol transport it seems unlikely that a possible alteration of the barrier properties should be caused by the treatment. Furthermore, all treatments were based on HBSS and this further indicates the unlikeliness of the treatments to have a harmful effect on the cell monolayers. A more likely explanation is that the monolayers were compromised physically (handling) during sampling.

LDH Assay

FIG. 14 shows the results of the LDH assay with absorbance at 492 nm as a measure for LDH release. In FIG. 4, the absorbance data is shown as a percentage relative to the absorbance obtained from cell monolayers exposed to supplemented HBSS (negative control). Elevated LDH release was only observed for the positive control (cells treated with ultrasound). The LDH release from IPEC-J2 MDR1 monolayers exposed to 10 μM solutions of the four compounds was comparable to HBSS. In absolute values, the absorbance measured from cell monolayers exposed to HBSS and the compound solutions where similar to background absorbance values (data not shown), which indicates no detectable amounts of LDH were released from these cell monolayers. Therefore, the observations from the LDH release assay indicate that HBSS and the four 10 μM drug solutions does not cause a disruption of the cell membranes.

So, the apical to basolateral transport experiment with 10 μM solutions of the four compounds showed that only Compound 1 was able to permeate through the cell monolayer. The appearance of Compound 2, 3, and 4 in the basolateral chamber was barely detectable. Increased mannitol transport was observed for one replicate treated with supplemented HBSS and for one replicate treated with 10 μM solution of Compound 1, which could indicate a disruption of the barrier properties. However, this was not reflected in the LDH release assay, which showed no negative effect of HBSS or the drug solutions on the integrity of the cell membranes.

Overall the findings of these experiments show that Compound 1 diffuses through IPEC-J2 MDR1 cell monolayers with a high permeability, while the permeability of Compound 2, 3 and 4 is low.

### Example 5

This experiment is conducted to assess the ability of four experimental compounds to lyse red blood cells in vitro. Since quaternary ammonium compounds tends to be toxic due to lysis, the inventors have tested the compounds against red blood cells.

Materials and Methods:

Materials

Bovine whole blood supplemented with citrate, Ultrapure water (MilliQ), Phosphate buffered Saline, 0.1% TritonX in PBS and Isotonic saline.

Experimental Compounds
Compound TH: 10-(2-(1-methylpiperidin-2-yl)ethyl)-2-(methylthio)-10H-phenothiazine hydrochloride (S23);
Compound TE: 1-ethyl-1-methyl-2-(2-(2-(methylthio)-1-OH-phenothiazin-10-yl)ethyl)piperidin-1-ium bromide (S25);
Compound TI: 1-isopentyl-1-methyl-2-(2-(2-(methylthio)-10H-phenothiazin-10-yl)ethyl)piperidin-1-ium bromide (S27);
Compound TP: 1-methyl-2-(2-(2-(methylthio)-10H-phenothiazin-10-yl)ethyl)-1-pentylpiperidin-1-ium bromide (S28).

The experimental compounds were dissolved in PBS to an initial concentration of 80 mg/L and treated with ultrasound. The initial stock solution was further diluted in PBS to produce solutions with concentrations of 1, 2 and 4 mg/L.

Red Blood Cell Lysis Assay

To investigate the ability of drug solutions to lyse red blood cells, 300 μL bovine whole blood was mixed with 1200 μl sample solution. The mixture of whole blood and sample solution was agitated for 30 minutes by end-over-end rotation at ambient temperatures.

Subsequently, the mixture was centrifuged for 10 minutes at 13000 RPM. The supernatant was diluted 20-fold in ultrapure water and the absorbance of the diluted supernatant at 540 nm was measured (LabSystems Multiscanner plate reader). All samples were measured in duplicate. PBS and isotonic saline was included as negative controls where no lysis of red blood cells is expected. Ultrapure water and a solution of 0.1% TritonX in PBS was included as positive controls where complete lysis of red blood cells is expected.

Results:

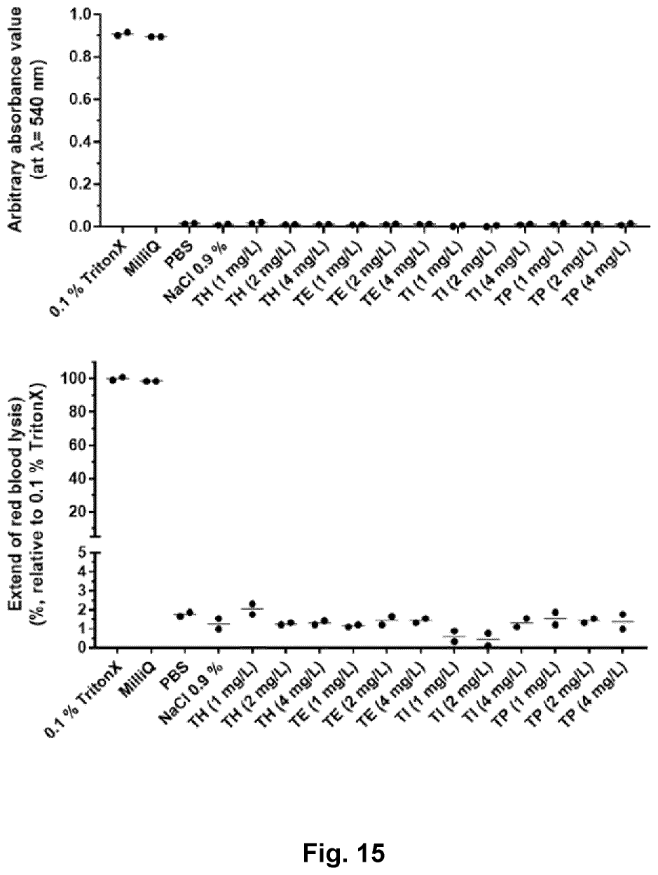

The results of the red blood cell lysis assay is shown in FIG. 15. The absorbance measured in the supernatant of whole blood incubated with solutions of the four compounds were negligible and comparable to the absorbance measured in the supernatant of whole blood incubated with PBS and isotonic saline (negative controls). The absorption measured in samples from the experimental compounds and the negative controls constitute ≥2% of the absorbance found in supernatants from cell monolayers exposed to 0.1 TritonX and MilliQ water (positive controls). TritonX is a surfactant and is thus able lyse cells by disrupting the cell membrane. Ultra pure water does practically not contain any solutes and are thus highly hypotonic. When cells are exposed to ultra pure water, e.g. MilliQ water, water will flow into the relative hypertonic cell cytosol and this will in turn cause cell rupture. It is therefore assumed that the lysis of cells exposed to either 0.1% TritonX and MilliQ water is complete (100%).

None of the experimental compounds, at concentrations of 1 mg/L, 2 mg/L and 4 mg/L, caused any increase in measured absorbance relative to phosphate buffered saline or isotonic saline (negative controls). It can therefore be concluded, that the tested compounds do not cause lysis of red blood cells in concentrations up to 4 mg/L.

### Example 6

This experiment is conducted to assess in vivo toxicity of the compounds. Compound S43 was selected for this study to evaluate for the highest tolerable dosage in mice. The study was performed at Statens Serum Institute. The Maximum Tolerated Dose (MTD) was investigated after intravenous (iv), intraperitonalt (ip) and peroral (po) dosing ranging from 5 mg/kg to 100 mg/kg. Mice were observed for clinical signs of discomfort for 4-6 hrs after injection. Compound S43 was tolerated after iv and ip dosing up to 10 mg/kg. Mice dosed twice po with 100 mg/kg showed no signs of discomfort as was thus considered well tolerated.

### Example 7

This experiment is conducted to assess the effect of topical treatment of the compounds against MRSA. Compound S43 was evaluated for efficacy against Staphylococcus aureus MRSA 43484 in an murine skin infection model. Treatment with compound S43 formulations at 1% and 2% resulted in significant reduction of the bacterial loads compared to vehicle treatment in the skin lesion.

Treatment was performed twice daily for 3 days and sampling of skin biopsies was performed the day after last treatment. Treatment with Fucidic acid was included as a positive control and treatment with vehicle was included as a negative control.

Materials and Methods

48 Balb/C female mice—18-22 g—Taconic, Denmark, Staphylococcus aureus MRSA43484, Test compound A at 1 and 2% formulations, Test compound B at 2% formulation, Fucidin, 0.9% saline (sterile, SSI), 0.9% saline/Triton-x (sterile, SSI), Sterile water (SSI), 5% Horse Blood Agar plates (SSI), MRSA Brilliance agar plates, Nurofen® Junior (20 mg/ml, Novartis), Zoletil mix/Torbugesic (SSI/QC-BIO), Dermal curette—Miltex 335710.

Laboratory Animal Facilities and Housing of Mice:

The temperature and humidity were registered daily in the animal facilities. The temperature was 22° C.+/−2° C. and can be regulated by heating and cooling. The humidity was 55+/−10%. The air changes per hour were approximately 8-12 times (70-73 times per hours inside racks), and light/dark period was in 12-hours interval of 6 a.m.-6 p.m./6 p.m-6 a.m. The mice had free access to domestic quality drinking water and food (Teklad Global diet 2916C-Envigo) and occasionally peanuts and sunflower seeds (Koge Korn A/S). The mice were housed in Type 3 macrolone cages with bedding from Tapvei. Further, the animals were offered Enviro-Dri nesting material and cardboard houses (Bio-serv).

Preparation of inoculum: Fresh overnight colonies from a 5% Horse Blood Agar plate were suspended in saline to approximately 109 CFU/ml.

Preparation of anaesthetic (Zoletil mix): Zoletil mix was diluted before use (total of 10 ml): 2 ml Zoletil mix+5.2 ml sterile saline+2.8 ml Torbugesic 1:100

Inoculation of Mice, Day 0

Approximately 1 hour before inoculation, mice were treated orally with 45 μl nurofen (20 mg ibuprofen/ml corresponding to approximately 30 mg/kg) as pain relief. The mice were anaesthetized with 0.15 ml s.c. of Zoletil mix. The fur was removed from a 2×3-cm area on the back of each mouse by use of an electric shaver. Next, a razor was used to remove all the hair and hereafter the outer most layer of the skin was scraped off with a dermal curette to obtain a 1 cm² superficial skin lesion. 10 μl inoculum containing approximately 107 CFU was spread on the lesion. After the applied inoculum had dried, the mouse was placed in the cage and kept in a warming cabinet until fully awake.

Dermal Treatment of Mice, Day 1-3

Topical treatment of was initiated the day after inoculation, Day 1. Mice were treated twice daily (9 a.m and 3 p.m.) for three days. A volume of 50 μl was spread on the inoculated skin area.

The mice were observed all days during study and scored 0-4 based on their behaviour and clinical signs.

| | |
|---|---|
| Score 0 | Healthy |
| Score 1 | Minor clinical signs of infection (slower movements) |
| Score 2 | Moderate signs of infection (lack of curiosity or changed |
| | activity) |
| Score 3 | Severe signs of infection (reduced movements, lightly |
| | pinched eyes) |
| Score 4 | Severe signs of infection (stiff movements, pinched eyes, |
| | cold,). Sacrificed. |

Sampling, Day 1 and Day 4

Colony counts in the skin lesions were determined on day 1 (start of treatment) and day 4 (the day after completed treatment). The mice were sacrificed day 1 and 4 according to Table 1. The affected skin area was removed by a pair of scissors and tweezers, and collected in a tube for Dispomixer with 1 ml saline. The skin sample was homogenized in a Dispomixer. Each sample was serial diluted in saline/Triton-x and 20-μl spots were applied on MRSA Brillince agar plates. All agar plates were incubated 20-48 hrs at 35° C.

Treatment Schedule.

| | | Day 1 | Day 4 |
|---|---|---|---|
| | Day 1-3 | sampling | sampling |
| Day 0 | Treatment | of skin | of skin |
| Inoculation | 0.05 ml/mouse | lesions | lesions |
| | Compound B 2% | | 1-2-3-4- |
| | | | 5-6-7-8 |
| MRSA | Compound A 2% | | 9-10-11-12- |
| | | | 13-14-15-16 |
| | Compound A 1% | | 17-18-19-20- |
| | | | 21-22-23-24 |
| | Fucidic acid | | 25-26-27-28- |
| | | | 29-30-31-32 |
| | Vehicle | | 33-34-35-36- |
| | | | 37-38-39-40 |
| | Start of treatment | 41-42-43-44- | |
| | | 45-46-47-48 | |

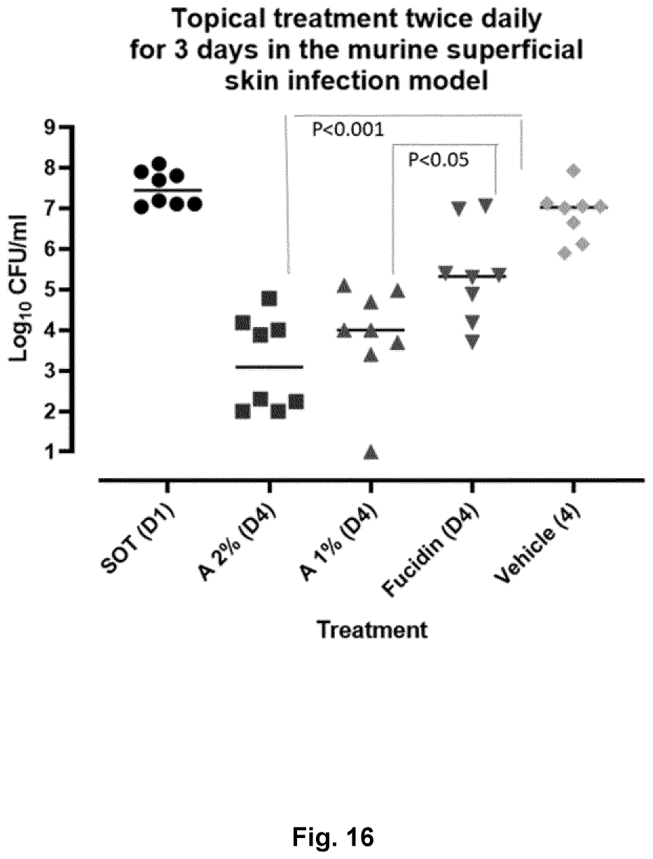

The colony count in the inoculum was determined to 8.76 log₁₀ CFU/ml, corresponding to 6.76 log₁₀ CFU/mouse. Colony counts in skin lesion were performed at day 1 (start of treatment) and day 4 post inoculation. The CFU counts are shown in FIG. 16. The CFU counts were log₁₀ transformed before performing calculations. No mice showed any clinical signs of infection or distress during the study.

Treatment with 1 and 2% test compound formulations resulted in a significant (p<0.0001; Anova; multiple comparisons) reduction of the bacterial loads of 3.0 and 3.7 log₁₀ CFU respectively compared to vehicle treatment in the skin lesion. Treatment with fucidic acid 2%, resulted in a 1.5 logo reduction of the CFU levels compared to the vehicle control (p<0.05).

A carry over effect was observed when counting colonies in spots of 10 fold dilutions of the skin samples. This indicates that a high concentration of active compound was still present in the skin biopsies at the time of sampling. This may in turn underestimate the number of viable bacteria detected on the agar plates for the samples where counting was performed in 10-100 fold dilutions.

### Example 8

The compound S43 was also assessed for mutagenicity. The results showed that S43 did not show indications of mutagenic potential (data not shown).

The invention is further described in the following non-limiting items.

Items
1. A composition comprising a compound of formula I

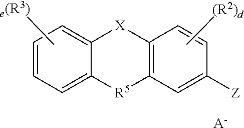

wherein
X is selected from the group consisting of S, Se, P, PO, SO, NR¹, CR¹, CR¹R¹ or C₀₋₂-alkyl;
Z is selected from the group consisting of hydrogen, a halogen, SR⁴, OR⁴, COR⁴ where R⁴ is a C₁₋₁₂-alkyl;
each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
d is selected from 0, 1, 2, and 3;
each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
e is selected from 0, 1, 2, 3, and 4;
R¹ is selected from the group consisting of C₁₋₆-alkyl, C₃₋₈-cycloalkyl, aryl, heteroaryl, arylalkyl, heteroarylalkyl;
R⁵ is N—(CHW)—N(Y¹)(Y²)(Y³) or C═CH—(CHW)—N(Y¹)(Y²)(Y³);
each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;
Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;
Y² is selected from the group consisting of C₁₋₁₂-alkyl;
Y³ is selected from the group consisting of linear or branched C₂₋₂₅-alkyl, linear or branched C₂₋₂₅ alkenyl or linear or branched C₂₋₂₅ alkynyl;

where A is selected from any pharmaceutical relevant/acceptable anion/counterion;

wherein if X is S and Z is a halogen then Y³ cannot be a C₂-alkyl or a branched C₃-alkyl;

wherein if X is S and Z is hydrogen then Y³ cannot be C₂-alkyl or linear or branched C₅-alkyl.
2. A composition according to item 1 wherein the compound of formula I is a phenothiazine derivative.
3. A composition according to item 2 wherein the phenothiazine derivative is selected from the group consisting of chlorpromazine derivatives, promethazine derivatives and thioridazine derivatives, and salts thereof.
4. A composition according to item 1 wherein the compound of formula I is a chlorprothixene derivative.
5. A composition according to item 3 wherein the compound of formula I is a thioridazine derivative.
6. A composition according to item 3 wherein the compound of formula I is a promethazine derivative.
7. A composition according to item 3 wherein the compound of formula I is a Chlorpromazine derivative.
8. A composition according to any of the preceding items wherein Y³ is a linear or branched C₂₋₆-alkyl.
9. A composition according to any of the preceding items wherein Y³ is selected from the group consisting of ethyl, propyl, methyl-butyl, iso-propyl or pentyl.
10. A composition according to any of the preceding items 1 to 7 wherein Y³ is a linear or branched C₈₋₁₅-alkyl.
11. A composition according to any of the preceding items 1 to 7 or 10 wherein Y³ is selected from the group consisting of linear or branched C₈-alkyl, linear or branched C₁₀-alkyl, linear or branched C₁₂-alkyl, linear or branched C₁₄-alkyl, linear or branched C₁₅-alkyl.
12. A composition according to any of the preceding items wherein Y¹ and Y² are individually selected from C₁₋₆-alkyl, such as from C₁₋₃-alkyl.
13. A composition according to any of the preceding items wherein Y¹ and Y² are both C₁-alkyl.
14. A composition according to any of the preceding items 1 to 9 wherein Y¹ together with a carbon being part of the W and the nitrogen atom to which it is attached forms a six-membered nitrogen-containing heterocyclyl.
15. A composition according to item 12 wherein Y² is selected from C₁₋₃-alkyl, such as C₁-alkyl.
16. A composition according to any of the preceding items wherein X is S.
17. A composition according to any of the preceding items wherein Z is selected from the group consisting of hydrogen, Cl or a SR⁴ where R⁴ is a C₁-alkyl.
18. An anti-microbial composition comprising a compound of formula I

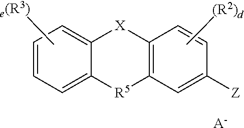

wherein
X is selected from the group consisting of S, Se, P, PO, SO, NR¹, CR¹, CR¹R¹ or C₀₋₂-alkyl;
Z is selected from the group consisting of hydrogen, a halogen, SR⁴, OR⁴, COR⁴ where R⁴ is a C₁₋₁₂-alkyl;
each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
d is selected from 0, 1, 2, and 3;
each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
e is selected from 0, 1, 2, 3, and 4;
R¹ is selected from the group consisting of C₁₋₆-alkyl, C₃₋₈-cycloalkyl, aryl, heteroaryl, arylalkyl, heteroarylalkyl;
R⁵ is N—(CHW)—N(Y¹)(Y²)(Y³) or C═CH—(CHW)—N(Y¹)(Y²)(Y³);
each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;
Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;
Y² is selected from the group consisting of C₁₋₁₂-alkyl;
Y³ is selected from the group consisting of linear or branched C₂₋₂₅-alkyl, linear or branched C₂₋₂₅ alkenyl or linear or branched C₂₋₂₅ alkynyl;
where A is selected from any pharmaceutical relevant/acceptable anion/counterion; for use as a medicament.
19. An anti-microbial composition for use according to item 16 wherein the compound of formula I is a phenothiazine derivative.
20. An anti-microbial composition for use according to item 17 wherein the phenothiazine derivative is selected from the group consisting of chlorpromazine derivatives, promethazine derivatives and thioridazine derivatives, and salts thereof.
21. An anti-microbial composition for use according to item 17 wherein the compound of formula I is a chlorprothixene derivative.
22. An anti-microbial composition for use according to item 18 wherein the compound of formula I is a thioridazine derivative.
23. An anti-microbial composition for use according to item 18 wherein the compound of formula I is a promethazine derivative.
24. An anti-microbial composition for use according to item 18 wherein the compound of formula I is a Chlorpromazine derivative.
25. An anti-microbial composition for use according to any of the preceding items 18 to 24 wherein Y³ is a linear or branched C₂₋₆-alkyl.
26. An anti-microbial composition for use according to any of the preceding items 18 to 25 wherein Y³ is selected from the group consisting of ethyl, propyl, methyl-butyl, iso-propyl or pentyl.
27. An anti-microbial composition for use according to any of the preceding items 18 to 24 wherein Y³ is a linear or branched C₈₋₁₅-alkyl.
28. An anti-microbial composition for use according to any of the preceding items 18 to 24 or 27 wherein Y³ is selected from the group consisting of linear or branched C₈-alkyl, linear or branched C₁₀-alkyl, linear or branched C₁₂-alkyl, linear or branched C₁₄-alkyl, linear or branched C₁₅-alkyl.
29. An anti-microbial composition for use according to any of the preceding items 18 to 28 wherein Y¹ and Y² are individually selected from C₁₋₆-alkyl, such as from C₁₋₃-alkyl.
30. An anti-microbial composition for use according to any of the preceding items 18 to 29 wherein Y¹ and Y² are both C₁-alkyl.
31. An anti-microbial composition for use according to any of the preceding items 18 to 28 wherein Y¹ together with a carbon being part of the Wand the nitrogen atom to which it is attached forms a six-membered nitrogen-containing heterocyclyl.
32. An anti-microbial composition for use according to item 31 wherein Y² is selected from C₁₋₃-alkyl, such as C₁-alkyl.
33. An anti-microbial composition for use to any of the preceding items 18 to 32 wherein X is S.
34. An anti-microbial composition for use according to any of the preceding items 18 to 33 wherein Z is selected from the group consisting of hydrogen, Cl or a SR⁴ where R⁴ is a C₁-alkyl.
35. An anti-microbial composition for use according to any of the preceding items 18 to 34 where the medicament is having antibacterial properties against resistant strains of bacteria.
36. An anti-microbial composition for use according to item 35 where the resistant strains (resistant towards conventional antimicrobial) of bacteria is selected from Staphylococcus, Bacillus, Enterococcus, Streptococcus, Listeria, Escherichia or Salmonella.
37. An anti-microbial composition for use according to any of the preceding items 18 to 36 where the medicament is having antibacterial properties against Gram-positive bacteria.
38. An anti-microbial composition for use according to item 37 wherein the Gram-positive bacteria is selected from Staphylococcus, Bacillus, Enterococcus, Streptococcus or Listeria.
39. An anti-microbial composition for use according to item 38 wherein the Gram-positive bacteria is selected from Staphylococcus aureus, Bacillus cereus, Enterococcus faecium, Enterococcus faecalis, Staphylococcus pseudintermedius, Streptococcus equi, Listeria monocytogenes.
40. An anti-microbial composition for use according to any of the preceding items 18 to 39 where the medicament is having antibacterial properties against multi-resistant strains and/or antimicrobial sensitive strains of bacteria.
41. An anti-microbial composition for use according to item 40 where the multi-resistant strains of bacteria is selected from Staphylococcus aureus or Bacillus cereus.
42. An anti-microbial composition for use according to any one of items 18 to 41 where the medicament has a minimum inhibitory concentration (MIC) below 16 μg/mL, such as below 8 μg/mL, such as below 4 μg/mL or such as below 2 μg/mL.
43. An anti-microbial composition for use according to any one of items 18 to 42 wherein if X is S and Z is a halogen then Y³ cannot be a C₂-alkyl or a branched C₃-alkyl; and/or wherein if X is S and Z is hydrogen then Y³ cannot be C₂-alkyl or linear or branched C₅-alkyl.
44. An anti-microbial composition comprising a compound of formula I

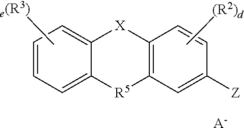

wherein
X is selected from the group consisting of S, Se, P, PO, SO, NR¹, CR¹, CR¹R¹ or C₀₋₂-alkyl;
Z is selected from the group consisting of hydrogen, a halogen, SR⁴, OR⁴, COR⁴ where R⁴ is a C₁₋₁₂alkyl;
each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
d is selected from 0, 1, 2, and 3;
each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
e is selected from 0, 1, 2, 3, and 4;
R¹ is selected from the group consisting of C₁₋₆-alkyl, C₃₋₈-cycloalkyl, aryl, heteroaryl, arylalkyl, heteroarylalkyl;
R⁵ is N—(CHW)—N(Y¹)(Y²)(Y³) or C═CH—(CHW)—N(Y¹)(Y²)(Y³);
each W is individually selected from the group consisting of linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;
Y¹ is selected from the group consisting of C₁₋₁₂-alkyl or together with the W and the nitrogen atom to which it is attached forms an optionally substituted nitrogen-containing heteroaryl or optionally substituted nitrogen-containing heterocyclyl;
Y² is selected from the group consisting of C₁₋₁₂-alkyl;
Y³ is selected from the group consisting of linear or branched C₂₋₂₅-alkyl, linear or branched C₂₋₂₅ alkenyl or linear or branched C₂₋₂₅ alkynyl;
where A is selected from any pharmaceutical relevant/acceptable anion/counterion; for use in treating a microbial infection in a human subject.
45. An anti-microbial composition for use according to item 44 wherein the compound of formula I is a phenothiazine derivative.
46. An anti-microbial composition for use according to item 45 wherein the phenothiazine derivative is selected from the group consisting of chlorpromazine derivatives, promethazine derivatives and thioridazine derivatives, and salts thereof.
47. An anti-microbial composition for use according to item 45 wherein the compound of formula I is a chlorprothixene derivative.
48. An anti-microbial composition for use according to item 46 wherein the compound of formula I is a thioridazine derivative.
49. An anti-microbial composition for use according to item 46 wherein the compound of formula I is a promethazine derivative.
50. An anti-microbial composition for use according to item 46 wherein the compound of formula I is a Chlorpromazine derivative.
51. An anti-microbial composition according to any of the preceding items 44 to 50 wherein Y³ is a linear or branched C₂₋₆-alkyl.
52. An anti-microbial composition for use according to any of the preceding items 44 to 51 wherein Y³ is selected from the group consisting of ethyl, propyl, methyl-butyl, iso-propyl or pentyl.
53. An anti-microbial composition for use according to any of the preceding items 44 to 50 wherein Y³ is a linear or branched C₈₋₁₅-alkyl.
54. An anti-microbial composition for use according to any of the preceding items 44 to 50 or 53 wherein Y³ is selected from the group consisting of linear or branched C₈-alkyl, linear or branched C₁₀-alkyl, linear or branched C₁₂-alkyl, linear or branched C₁₄-alkyl, linear or branched C₁₅-alkyl.
55. An anti-microbial composition for use according to any of the preceding items 44 to 54 wherein Y¹ and Y² are individually selected from C₁₋₆-alkyl, such as from C₁₋₃-alkyl.
56. An anti-microbial composition for use according to any of the preceding items 44 to 55 wherein Y¹ and Y² are both C₁-alkyl.
57. An anti-microbial composition for use according to any of the preceding items 44 to 54 wherein Y¹ together with a carbon being part of the Wand the nitrogen atom to which it is attached forms a six-membered nitrogen-containing heterocyclyl.
58. An anti-microbial composition for use according to item 57 wherein Y² is selected from C₁₋₃-alkyl, such as C₁-alkyl.
59. An anti-microbial composition for use to any of the preceding items 44 to 58 wherein X is S.
60. An anti-microbial composition for use according to any of the preceding items 44 to 59 wherein Z is selected from the group consisting of hydrogen, Cl or a SR⁴ where R⁴ is a C₁-alkyl.
61. An anti-microbial composition for use according to any of the preceding items 44 to 60 where the medicament is having antibacterial properties against resistant strains of bacteria.
62. An anti-microbial composition for use according to item 60 where the resistant strains (resistant towards conventional antimicrobial) of bacteria is selected from Staphylococcus, Bacillus, Enterococcus, Streptococcus, Listeria, Escherichia or Salmonella.
63. An anti-microbial composition for use according to any of the preceding items 44 to 62 where the medicament is having antibacterial properties against Gram-positive bacteria.
64. An anti-microbial composition for use according to item 63 wherein the Gram-positive bacteria is selected from Staphylococcus, Bacillus, Enterococcus, Streptococcus or Listeria.
65. An anti-microbial composition for use according to item 64 wherein the Gram-positive bacteria is selected from Staphylococcus aureus, Bacillus cereus, Enterococcus faecium, Enterococcus faecalis, Staphylococcus pseudintermedius, Streptococcus equi, Listeria monocytogenes.
66. An anti-microbial composition for use according to any of the preceding items 44 to 65 where the medicament is having antibacterial properties against multi-resistant strains of bacteria.
67. An anti-microbial composition for use according to item 66 where the multi-resistant strains of bacteria is selected from Staphylococcus aureus or Bacillus cereus.
68. An anti-microbial composition for use according to any one of items 44 to 67 where the medicament has a minimum inhibitory concentration (MIC) below 16 μg/mL, such as below 8 μg/mL, such as below 4 μg/mL or such as below 2 μg/mL.
69. An anti-microbial composition for use according to any one of items 44 to 68 wherein if X is S and Z is a halogen then Y³ cannot be a C₂-alkyl or a branched C₃-alkyl; and/or wherein if X is S and Z is hydrogen then Y³ cannot be C₂-alkyl or linear or branched C₅-alkyl.

1. A composition comprising a compound of formula I: 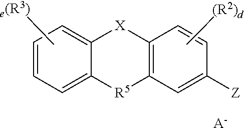
wherein:
X is selected from the group consisting of S, Se, P, PO, SO, NR¹, CR¹, CR¹R¹ and C₀₋₂-alkyl;
Z is selected from the group consisting of hydrogen, a halogen, SR⁴, OR⁴, and COR⁴ where R⁴ is a C₁₋₁₂-alkyl;
each R² is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
d is selected from 0, 1, 2, or 3;
each R³ is independently selected from the group consisting of C₁₋₆-alkyl, halogen, C₃₋₈-cycloalkyl, OH, NH₂, NHR¹, N(R¹)₂, O—C₁₋₆-alkyl, O—C₃₋₈-cycloalkyl, NH—C₁₋₆-alkyl, NH—C₃₋₈-cycloalkyl, S—C₁₋₆-alkyl, S—C₃₋₈-cycloalkyl, aryl, heteroaryl, aryloxy, heteroaryloxy, arylamino, heteroarylamino, arylalkyl, heteroarylalkyl, arylalkyloxy and heteroarylalkyloxy;
e is selected from 0, 1, 2, 3, or 4;
R¹ is selected from the group consisting of C₁₋₆-alkyl, C₃₋₈-cycloalkyl, aryl, heteroaryl, arylalkyl, and heteroarylalkyl;
R⁵ is N—(CHW)—N(Y¹)(Y²)(Y³) or C═CH—(CHW)—N(Y¹)(Y²)(Y³);
each W is individually selected from linear or branched C₁₋₆-alkyl or together with the nitrogen atom —N(Y¹)(Y²)(Y³)— to which it is attached forms an optionally substituted nitrogen-containing heteroaryl or optionally substituted nitrogen-containing heterocyclyl together with Y¹ where;
Y¹ is selected from C₁₋₁₂-alkyl or together with the W and the nitrogen atom to which it is attached forms an optionally substituted nitrogen-containing heteroaryl or optionally substituted nitrogen-containing heterocyclyl;
Y² is selected from C₁₋₁₂-alkyl;
Y³ is selected from linear or branched C₂₋₂₅-alkyl, linear or branched C₂₋₂₅ alkenyl or linear or branched C₂₋₂₅ alkynyl;
where A is selected from any pharmaceutical acceptable anion or counterion;
wherein if X is S and Z is a halogen then Y³ cannot be a C₂-alkyl or a branched C₃-alkyl; and
wherein if X is S and Z is hydrogen then Y³ cannot be C₂-alkyl or linear or branched C₅-alkyl.

2-32. (canceled)

33. The composition according to claim 1 wherein the compound of formula I is a phenothiazine derivative selected from the group consisting of chlorpromazine derivatives, promethazine derivatives and thioridazine derivatives, and salts thereof or wherein the compound of formula I is a chlorprothixene derivative.

34. The composition according to claim 1 wherein Y³ is a linear or branched C₅₋₂₅-alkyl.

35. The composition according to claim 1 wherein Y³ is a linear or branched C₂₋₆-alkyl.

36. The composition according to claim 1 wherein Y¹ and Y² are individually selected from C₁₋₆-alkyl.

37. The composition according to claim 1 wherein Y¹ and Y² are both C₁-alkyl.

38. The composition according to claim 1 wherein Y¹ together with a carbon being part of the W and the nitrogen atom to which it is attached forms a six-membered nitrogen-containing heterocyclyl.

39. The composition according to claim 1 wherein Z is selected from hydrogen, Cl or a SR⁴ where R⁴ is a C₁-alkyl.

40. A method of inhibiting a microbial infection in a subject comprising:
administering the composition of claim 1 to a subject that has a microbial infection.

41. The method according to claim 40, wherein the compound of formula I is a phenothiazine derivative selected from the group consisting of chlorpromazine derivatives, promethazine derivatives and thioridazine derivatives, and salts thereof or wherein the compound of formula I is a chlorprothixene derivative.

42. The method according to claim 40, wherein Y³ is a linear or branched C₅₋₂₅-alkyl.

43. The method according to claim 40, wherein Y³ is a linear or branched C₂₋₆-alkyl.

44. The method according to claim 40, wherein the microbial infection comprises a resistant strain of bacteria selected from Staphylococcus, Bacillus, Enterococcus, Streptococcus, Listeria, Escherichia or Salmonella.

45. The method according to claim 40, wherein composition provides a minimum inhibitory concentration (MIC) below 16 μg/mL.

46. The composition of claim 34 wherein Y³ is a linear or branched C₈₋₁₅-alkyl.

47. The composition of claim 34 wherein Y³ is selected from the group consisting of a linear or branched C₈-alkyl, a linear or branched C₁₀-alkyl, a linear or branched C₁₂-alkyl, a linear or branched C₁₄-alkyl, and a linear or branched C₁₅-alkyl.

48. The composition of claim 35 wherein Y³ is a linear or branched C₂₋₆-alkyl selected from the group consisting of ethyl, propyl, methyl-butyl, iso-propyl and pentyl.

49. The method of claim 42 wherein Y³ is a linear or branched C₈₋₁₅-alkyl.

50. The method of claim 42 wherein Y³ is selected from the group consisting of a linear or branched C₈-alkyl, a linear or branched C₁₀-alkyl, a linear or branched C₁₂-alkyl, a linear or branched C₁₄-alkyl, and a linear or branched C₁₅-alkyl.

51. The method of claim 43 wherein Y³ is a linear or branched C₂₋₆-alkyl selected from the group consisting of ethyl, propyl, methyl-butyl, iso-propyl and pentyl.
